(19) United States
(12) Patent Application Publication
(10) Pub. No.: US 2025/0268512 A1
(43) Pub. Date: Aug. 28, 2025

(54) SYSTEMS AND METHODS FOR ASSESSING SPECTRAL DATA CORRESPONDING TO ELECTROMYOGRAPHIC SIGNALS OF THE GASTROINTESTINAL TRACT

(71) Applicant: G-Tech Medical, Inc., Mountain View, CA (US)

(72) Inventors: Steve Axelrod, Los Altos, CA (US); Anand Navalgund, San Jose, CA (US); Lindsay Axelrod, Los Altos, CA (US)

(21) Appl. No.: 19/064,101

(22) Filed: Feb. 26, 2025

Related U.S. Application Data
(60) Provisional application No. 63/558,914, filed on Feb. 28, 2024.

(51) Int. Cl. A61B 5/397; A61B 5/00; A61B 5/257; A61B 5/296; A61B 5/392

(52) U.S. Cl. CPC A61B 5/397; A61B 5/257; A61B 5/296; A61B 5/392; A61B 5/4238; A61B 5/4255; A61B 5/6823; A61B 5/7203; A61B 5/7221; A61B 5/7246; A61B 5/7264; A61B 2560/0468

(57) ABSTRACT

A system and a method for analyzing spectral peaks associated with movement in a gastrointestinal tract, the method comprising: determining spectral data from electromyographic data originating from smooth muscles associated with one or more organs of the gastrointestinal tract; executing a mathematical fit of the spectral data based on at least one shaping function; identifying, based on the executed mathematical fit, one or more candidate peaks in the spectral data for a time interval; determining, for the one or more candidate peaks, a plurality of parameters that quantify underlying rhythmic activity of one or more gastrointestinal organ of the gastrointestinal tract; and selecting, based on the determined plurality of parameters and the mathematical fit, at least one of the one or more candidate peaks to represent valid activity of the gastrointestinal tract during the time interval.

### CROSS-REFERENCE TO RELATED APPLICATION

This application is a continuation of U.S. Provisional Application No. 63/558,914, titled “Systems and Methods for Assessing Spectral Data Corresponding to Electromyographic Signals of the Gastrointestinal Tract,” filed Feb. 28, 2024, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to systems and methods for capturing and analyzing electrical activity within the smooth muscle of the gastrointestinal tract, and more particularly to systems and methods for processing electronic time series recordings from electromyographic activity of the gastrointestinal tract.

### BACKGROUND

Obtaining electromyography (EMG) data from the gastrointestinal tract of a person conventionally includes a procedure performed in a clinical setting, within a time frame of several hours, and wherein the patient is substantially in repose. The procedure may also include pre-procedure guidelines to ensure the person adhere to a standardized schedule of eating, and to eating a standardized meal. These constraints, however practical and appropriate, nevertheless can limit the scope of data derived from such studies. The data are limited in time frame, that is, conventional studies performed in the above manner are feasible for several hours, during which a patient can tolerate or comply with the constraint on normal physical activity. This limitation can be understood from the perspective that gastrointestinal activity occurs in the context of a daily cycle, and that daily cycle occurs in the context of activities of daily living. Gastrointestinal pain or discomfort also can be cyclical or intermittent throughout the day, or over the course of several days. Such intermittency may or may not be clearly tied to activities associated with the gastrointestinal tract specifically or the more general and varied activities of daily living. Accordingly, signals and patterns of interest may not present themselves in the limited observational window of the clinical test; thus, the diagnostic value of gastrointestinal activity data derived from tests that include such constraints is limited.

### SUMMARY

Systems and methods are described herein that relate to a method for analyzing spectral peaks associated with movement in a gastrointestinal tract, the method including: determining spectral data from electromyographic data originating from smooth muscles associated with one or more organs of the gastrointestinal tract; executing a mathematical fit of the spectral data based on at least one shaping function; identifying, based on the executed mathematical fit, one or more candidate peaks in the spectral data for a time interval; determining, for the one or more candidate peaks, a plurality of parameters that quantify underlying rhythmic activity of one or more gastrointestinal organ of the gastrointestinal tract; and selecting, based on the determined plurality of parameters and the mathematical fit, at least one of the one or more candidate peaks to represent valid activity of the gastrointestinal tract during the time interval.

In some aspects, the techniques described herein relate to a method, wherein the plurality of parameters include: a center frequency of the one or more candidate peaks, a baseline value of the one or more candidate peaks, a peak width of the one or more candidate peaks, an amplitude of the one or more candidate peaks, and a height of the peak above the baseline value associated with the respective one or more candidate peaks.

In some aspects, the techniques described herein relate to a method, wherein the time interval ranges from about two minutes to about 4 days.

In some aspects, the techniques described herein relate to a method, wherein the spectral data represents time series data obtained from one or more cutaneous patches placed on an abdominal region of a subject.

In some aspects, the techniques described herein relate to a method, wherein the shaping function is a Gaussian function, a Lorentzian function, or other substantially bell-shaped function.

In some aspects, the techniques described herein relate to a method, wherein the one or more gastrointestinal organ includes at least one of: a stomach, a small intestine, and a colon.

In some aspects, the techniques described herein relate to a method, wherein executing the mathematical fit includes: setting, for the spectral data, a first threshold applicable to identifying an approximate amplitude or width of the one or more candidate peaks; setting, for the spectral data, a second threshold applicable for identifying an approximate frequency of the one or more candidate peaks; and using the identified approximate amplitude or approximate width and the approximate frequency as input into the mathematical fit to identify the one or more candidate peaks, wherein both the first threshold and the second threshold are determined based on values within a frequency spectrum associated with the spectral data.

In some aspects, the techniques described herein relate to a method, wherein identifying the one or more candidate peaks in the spectral data further includes identifying points within the spectral data that are above the second threshold by executing one or more of: a peak detector that imposes constraints including consecutive values above the first threshold, and a peak detector that provides smoothing prior to identifying points within the spectral data that are above the second threshold.

In some aspects, the techniques described herein relate to a method, wherein executing the mathematical fit of the spectral data includes generating, for the spectral data, an optimized background signal associated with the one or more candidate peaks, the optimized background signal resulting in noise reduction of the spectral data in the time interval.

In some aspects, the techniques described herein relate to a method, wherein: a first 1 to identify a second of the one or more candidate peaks.

In some aspects, the techniques described herein relate to a method, further including: removing each of the one or more candidate peaks from the spectral data resulting in background signal; generating an average value range of the background signal; determining a difference between the average value range and a predefined average background level; generating, based on the determined difference, a normalization factor corresponding to one or more physiological features; applying the normalization factor to the spectral data associated with subjects exhibiting one or more of the physiological features, wherein the normalization factor corrects the mathematical fit according to the one or more physiological features.

In some aspects, the techniques described herein relate to a method, wherein the one or more physiological features are selected from at least one of: subject girth, subject skin condition, subject health condition, subject muscle condition, and subject gastrointestinal tract anomalies.

In some aspects, the techniques described herein relate to a method, wherein the method is iteratively performed and each iteration uses a different fitting techniques optimized for identifying candidate peaks having differing widths.

In some aspects, the techniques described herein relate to a method, wherein the fitting techniques include one or more of: a spectrum smoothing filter, a peak width range thresholding filter, and a Gaussian fitting.

In some aspects, the techniques described herein relate to a method, wherein: the spectral data includes multiple channels and represents time series data obtained from at least two sets of electrodes placed on an abdominal region of a subject and configured to simultaneously capture data; and for each set of electrodes the method further includes: executing the mathematical fit of the spectral data based on the at least one shaping function; identifying, based on the executed mathematical fit, a set of candidate peaks in the spectral data for a time interval; determining, for the set of candidate peaks, a plurality of parameters that quantify underlying rhythmic activity; and selecting, based on the determined plurality of parameters and the mathematical fit, at least one of the set of candidate peaks; comparing the selected set of candidate peaks of a first set of electrodes, in the at least two sets of electrodes, to the selected set of candidate peaks of a second set of electrodes in the at least two sets of electrodes; in response to determining, based on the comparison, that the selected set of candidate peaks of the first set of electrodes and the selected set of candidate peaks of the second set of electrodes appear within the time interval on two or more of the multiple channels, increasing a confidence level that the selected sets of candidate peaks represent valid activity of the gastrointestinal tract.

In some aspects, the techniques described herein relate to a method for analyzing spectral peaks associated with movement in a gastrointestinal tract, the method including: obtaining time series data from a skin-surface mounted electrode patch configured to sense and acquire EMG voltage signals associated with movement in the gastrointestinal tract, the time series data being obtained for a plurality of time segments over a plurality of channels; for each respective time segment: identifying a first set of candidate peaks in the time series data using a first cleanup level; identifying a second set of candidate peaks in the time series data using a second cleanup level; identifying a third set of candidate peaks in the time series data using a third cleanup level; comparing, for each time segment in the plurality of time segments, peaks in the first set of candidate peaks to peaks in the second set of candidate peaks, and peaks in the third set of candidate peaks; and selecting, for each cleanup level and based on the comparison and a predefined parameter, at least one of the peaks from the first, second, or third sets of candidate peaks as an optimized peak for the respective time segment.

In some aspects, the techniques described herein relate to a method, wherein the predefined parameter includes: a maximum peak amplitude, a maximum peak area, a minimum peak width, or a minimum Gaussian residual.

In some aspects, the techniques described herein relate to a method, wherein: the first cleanup level is about 100,000 analog-to-digital converter (ADC) counts; the second cleanup level is about 50,000 ADC counts; and the third cleanup level is about 35,000 ADC counts.

In some aspects, the techniques described herein relate to a method, further including for each respective time segment: identifying a fourth set of candidate peaks in the time series data using a fourth cleanup level; identifying a fifth set of candidate peaks in the time series data using a fifth cleanup level; identifying a sixth set of candidate peaks in the time series data using a sixth cleanup level; comparing, for each time segment in the plurality of time segments, peaks in the first set of candidate peaks to peaks in the second set of candidate peaks, peaks in the third set of candidate peaks, peaks in the fourth set of candidate peaks, peaks in the fifth set of candidate peaks, and peaks in the sixth set of candidate peaks; and selecting, for each cleanup level and based on the comparison and the predefined parameter, at least one of the peaks from the first, second, third, fourth, fifth, or sixth, sets of candidate peaks as an optimized peak for the respective time segment.

In some aspects, the techniques described herein relate to a method, wherein: the first cleanup level is about 100,000 analog-to-digital converter (ADC) counts; the second cleanup level is about 50,000 ADC counts; the third cleanup level is about 35,000 ADC counts; the fourth cleanup level is about 20,000 ADC counts; the fifth cleanup level is about 10,000 ADC counts; and the sixth cleanup level is about 5,000 ADC counts.

In some aspects, the techniques described herein relate to a method, further including: generating a normalization factor for at least one of the first, second, or third set of candidate peaks; normalizing an amplitude feature in the time series data by applying the generated normalization factor to the time series data.

In some aspects, the techniques described herein relate to a system for analyzing spectral peaks associated with movement in a gastrointestinal tract, the system including: at least one electrode patch mounted on a skin surface of a patient; at least one processor communicatively coupled to the at least one patch, the at least one processor being configured to characterize parameters of peaks in a frequency spectrum of a gastrointestinal EMG data set acquired from the at least one electrode patch, the characterization including: determining spectral data from electromyographic data captured by the at least one electrode patch and originating from smooth muscles associated with one or more organs of the gastrointestinal tract; executing a mathematical fit of the spectral data based on at least one shaping function; identifying, based on the executed mathematical fit, one or more candidate peaks in the spectral data for a time interval; determining, for the one or more candidate peaks, a plurality of parameters that quantify underlying rhythmic activity of one or more gastrointestinal organ of the gastrointestinal tract; and selecting, based on the determined plurality of parameters and the mathematical fit, at least one of the one or more candidate peaks to represent valid activity of the gastrointestinal tract during the time interval.

In some aspects, the techniques described herein relate to a system, wherein the plurality of parameters include: a center frequency of the one or more candidate peaks, a baseline value of the one or more candidate peaks, a peak width of the one or more candidate peaks, an amplitude of the one or more candidate peaks, and a height of the peak above the baseline value associated with the respective one or more candidate peaks.

In some aspects, the techniques described herein relate to a system, wherein the time interval ranges from about two minutes to about 4 days.

In some aspects, the techniques described herein relate to a system, wherein the spectral data represents time series data obtained from one or more cutaneous patches placed on an abdominal region of a subject.

In some aspects, the techniques described herein relate to a system, wherein the shaping function is a Gaussian function, a Lorentzian function, or other substantially bell-shaped function.

In some aspects, the techniques described herein relate to a system, wherein the one or more gastrointestinal organ includes at least one of: a stomach, a small intestine, and a colon.

In some aspects, the techniques described herein relate to a system, wherein executing the mathematical fit includes: setting, for the spectral data, a first threshold applicable to identifying an approximate amplitude or width of the one or more candidate peaks; setting, for the spectral data, a second threshold applicable for identifying an approximate frequency of the one or more candidate peaks; and using the identified approximate amplitude or approximate width and the approximate frequency as input into the mathematical fit to identify the one or more candidate peaks, wherein both the first threshold and the second threshold are determined based on values within a frequency spectrum associated with the spectral data.

In some aspects, the techniques described herein relate to a system, wherein identifying the one or more candidate peaks in the spectral data further includes identifying points within the spectral data that are above the second threshold by executing one or more of: a peak detector that imposes constraints including consecutive values above the first threshold, and a peak detector that provides smoothing prior to identifying points within the spectral data that are above the second threshold.

In some aspects, the techniques described herein relate to a system, wherein executing the mathematical fit of the spectral data includes generating, for the spectral data, an optimized background signal associated with the one or more candidate peaks, the optimized background signal resulting in noise reduction of the spectral data in the time interval. In some aspects, the techniques described herein relate to a system, wherein: a first 1 to identify a second of the one or more candidate peaks.

In some aspects, the techniques described herein relate to a system, further including: removing each of the one or more candidate peaks from the spectral data resulting in background signal; generating an average value range of the background signal; determining a difference between the average value range and a predefined average background level; generating, based on the determined difference, a normalization factor corresponding to one or more physiological features; applying the normalization factor to the spectral data associated with subjects exhibiting one or more of the physiological features, wherein the normalization factor corrects the mathematical fit according to the one or more physiological features.

In some aspects, the techniques described herein relate to a system, wherein the one or more physiological features are selected from at least one of: subject girth, subject skin condition, subject health condition, subject muscle condition, and subject gastrointestinal tract anomalies.

In some aspects, the techniques described herein relate to a system, further including iteratively performing the determining of the spectral data, the execution of the mathematical fit, the identifying of the one or more candidate peaks, the determining of the plurality of parameters, and the selecting of at least one of the one or more candidate peaks, wherein each iteration uses a different fitting technique optimized for identifying candidate peaks having differing widths.

In some aspects, the techniques described herein relate to a system, wherein the fitting techniques include one or more of: a spectrum smoothing filter, a peak width range thresholding filter, and a Gaussian fitting.

In some aspects, the techniques described herein relate to a system, wherein: the spectral data includes multiple channels and represents time series data obtained from at least two sets of electrodes placed on an abdominal region of a subject and configured to simultaneously capture data; and for each set of electrodes the method further includes: executing the mathematical fit of the spectral data based on the at least one shaping function; identifying, based on the executed mathematical fit, a set of candidate peaks in the spectral data for a time interval; determining, for the set of candidate peaks, a plurality of parameters that quantify underlying rhythmic activity; and selecting, based on the determined plurality of parameters and the mathematical fit, at least one of the set of candidate peaks; comparing the selected set of candidate peaks of a first set of electrodes, in the at least two sets of electrodes, to the selected set of candidate peaks of a second set of electrodes in the at least two sets of electrodes; in response to determining, based on the comparison, that the selected set of candidate peaks of the first set of electrodes and the selected set of candidate peaks of the second set of electrodes appear within the time interval on two or more of the multiple channels, increasing a confidence level that the selected sets of candidate peaks represent valid activity of the gastrointestinal tract.

In some aspects, the techniques described herein relate to a system for analyzing spectral peaks associated with movement in a gastrointestinal tract, the system including: at least one electrode patch mounted on a skin surface of a patient; at least one processor communicatively coupled to the at least one patch, the at least one processor being configured to characterize parameters of peaks in a frequency spectrum of a gastrointestinal EMG data set acquired from the at least one electrode patch, the characterization including: obtaining time series data from a skin-surface mounted electrode patch configured to sense and acquire EMG voltage signals associated with movement in the gastrointestinal tract, the time series data being obtained for a plurality of time segments over a plurality of channels; for each respective time segment: identifying a first set of candidate peaks in the time series data using a first cleanup level; identifying a second set of candidate peaks in the time series data using a second cleanup level; identifying a third set of candidate peaks in the time series data using a third cleanup level; comparing, for each time segment in the plurality of time segments, peaks in the first set of candidate peaks to peaks in the second set of candidate peaks, and peaks in the third set of candidate peaks; and selecting, for each cleanup level and based on the comparison and a predefined parameter, at least one of the peaks from the first, second, or third sets of candidate peaks as an optimized peak for the respective time segment.

In some aspects, the techniques described herein relate to a system, wherein the predefined parameter includes: a maximum peak amplitude, a maximum peak area, a minimum peak width, or a minimum Gaussian residual. In some aspects, the techniques described herein relate to a method, wherein: the first cleanup level is about 100,000 analog-to-digital converter (ADC) counts; the second cleanup level is about 50,000 ADC counts; and the third cleanup level is about 35,000 ADC counts.

In some aspects, the techniques described herein relate to a system, further including for each respective time segment: identifying a fourth set of candidate peaks in the time series data using a fourth cleanup level; identifying a fifth set of candidate peaks in the time series data using a fifth cleanup level; identifying a sixth set of candidate peaks in the time series data using a sixth cleanup level; comparing, for each time segment in the plurality of time segments, peaks in the first set of candidate peaks to peaks in the second set of candidate peaks, peaks in the third set of candidate peaks, peaks in the fourth set of candidate peaks, peaks in the fifth set of candidate peaks, and peaks in the sixth set of candidate peaks; and selecting, for each cleanup level and based on the comparison and the predefined parameter, at least one of the peaks from the first, second, third, fourth, fifth, or sixth, sets of candidate peaks as an optimized peak for the respective time segment.

In some aspects, the techniques described herein relate to a system, wherein: the first cleanup level is about 100,000 analog-to-digital converter (ADC) counts; the second cleanup level is about 50,000 ADC counts; the third cleanup level is about 35,000 ADC counts; the fourth cleanup level is about 20,000 ADC counts; the fifth cleanup level is about 10,000 ADC counts; and the sixth cleanup level is about 5,000 ADC counts.

In some aspects, the techniques described herein relate to a system, further including: generating a normalization factor for at least one of the first, second, or third set of candidate peaks; normalizing an amplitude feature in the time series data by applying the generated normalization factor to the time series data.

In some aspects, the techniques described herein relate to a method including: receiving a plurality of spectrum data sets, wherein each spectrum data set corresponds to a frequency analysis of a time segment of a plurality of time segments of gastrointestinal myoelectrical signals, wherein each spectrum data set extends across a range of frequencies, defining a set of cleanup levels, and for each cleanup level of the set of cleanup levels, and for each spectrum data set: identify peaks in the spectrum data set, wherein the identified peaks correspond to spectrum data elements that satisfy one or more criteria based on the cleanup level, wherein each identified peaks is associated with a frequency in the range of frequencies, store the identified peaks in a peak data set associated with the spectrum data set and cleanup level, and remove the identified peaks from the spectrum data set; and identifying a plurality of sub-bands within the range of frequency, and for each sub-band of the plurality of sub-bands: identify a peak having a highest amplitude in the respective sub-band based on the identified peaks in the peak data sets that are within the respective sub-band, and store the identified peak in a composite result file associated with the plurality of spectrum data sets; and providing information to a user based on the composite result file.

In some aspects, the techniques described herein relate to a method, further including: determining, based on the composite result file and predefined organ activity definitions, which organ emitted the identified peak.

In some aspects, the techniques described herein relate to a method, wherein identifying the plurality of sub-bands includes identifying clusters of identified peaks within the peak data sets.

In some aspects, the techniques described herein relate to a method, wherein the one or more criteria include, for each cleanup level, at least one of: a threshold peak height, a threshold peak width, and a threshold peak energy.

In some aspects, the techniques described herein relate to a method, wherein determining the identified peak is based on at least one of: a largest peak height, a largest peak energy, a narrowest peak width, and a lowest Gaussian residual.

In some aspects, the techniques described herein relate to a method, wherein the identified peak is a composite of some or all of the identified peaks within the sub-band.

In some aspects, the techniques described herein relate to a method, including normalizing the plurality of spectrum data sets.

In some aspects, the techniques described herein relate to a method, wherein normalizing the plurality of spectrum data sets includes determining one or more normalization factors based on an analysis of a select frequency band of the spectrum data sets.

In some aspects, the techniques described herein relate to a method, wherein the one or more normalization factors are based on the select frequency band and a given cleanup level.

In some aspects, the techniques described herein relate to a method, including: determining a noise floor associated with each spectrum data set of the plurality of spectrum data sets, and removing the noise floor from each spectrum data set.

In some aspects, the techniques described herein relate to a method, wherein determining the noise floor associated with each spectrum data set includes determining a common noise floor associated with the plurality of spectrum data sets.

In some aspects, the techniques described herein relate to a method, wherein determining the identified peak includes excluding any identified peak that does not appear in a minimum number of peak data sets.

In some aspects, the techniques described herein relate to a method, further including: performing the frequency analysis of the time segments to provide the plurality of spectrum data sets.

In some aspects, the techniques described herein relate to a method, further including: determining a mathematical fit of the identified peak to a characteristic function, wherein determining the mathematical fit includes determining parameters of the characteristic function to create a parameterized characteristic function, and using the parameterized characteristic function to define the identified peak.

In some aspects, the techniques described herein relate to a method, wherein the characteristic function includes a Gaussian distribution function.

In some aspects, the techniques described herein relate to a method, wherein the plurality of time segments of gastrointestinal myoelectrical signals is obtained from at least one skin-surface mounted electrode patch.

In some aspects, the techniques described herein relate to a method, wherein the at least one skin-surface mounted electrode patch includes a plurality of channels, wherein each channel provides a portion of the plurality of time segments of gastrointestinal myoelectrical signals.

In some aspects, the techniques described herein relate to a method, wherein each time segment is between about 8 and about 12 minutes.

In some aspects, the techniques described herein relate to a method including: obtaining cardiac-determined information based on first sensor signals that indicate cardiac activity over a time period, wherein the cardiac-determined information includes values of one or more cardiac-determined attributes over the time period, wherein the cardiac-determined attributes include at least one of: a heart rate, a heart rate variability, an awake/asleep state, an active/rest state, and a stress level; obtaining gastric-determined information based on second sensor signals that indicate gastric activity over the time period, wherein the gastric-determined information includes values of one or more gastric-determined attributes over the time period, wherein the gastric-determined attributes include one or more of: gastric motility, strength of gastric muscle activity, frequency of gastric muscle activity, a gastric state, and a gastric disorder; and concurrently presenting the cardiac-determined information and gastric-determined information to a user, wherein the presenting of the cardiac-determined information and gastric-determined information includes presenting at least one of: textual information and graphic information.

In some aspects, the techniques described herein relate to a method, wherein: the first sensor signals and the second sensor signals are each derived from a same set of sensor signals.

In some aspects, the techniques described herein relate to a method, wherein the same sensor signals are obtained from a device including a skin-surface mounted electrode patch.

In some aspects, the techniques described herein relate to a method, wherein the same sensor signals are obtained with a sampling rate below about 5 Hertz.

In some aspects, the techniques described herein relate to a method, wherein the skin-surface mounted electrode patch includes a plurality of channels, wherein each channel provides a portion of the same sensor signals.

In some aspects, the techniques described herein relate to a method, wherein the second sensor signals are obtained with a sampling rate below 10 Hertz.

In some aspects, the techniques described herein relate to a method, including: determining one or more correlations between the cardiac-determined attributes and the gastric-determined attributes; and presenting, to the user, correlation information based on the one or more correlations.

In some aspects, the techniques described herein relate to a method, wherein the correlation information includes a rate of change of values of at least one gastric-determined attribute as a function of changes of values of at least one cardiac-determined attribute.

In some aspects, the techniques described herein relate to a method, wherein the rate of change is presented as at least one of: a ratio of a first value of the gastric-determined attribute and a second value of the gastric-determined attribute; and a percentage change of the first value of the gastric-determined attribute relative to the second value of the gastric-determined attribute.

In some aspects, the techniques described herein relate to a method, wherein the gastric-determined attributes include muscle activity associated with each gastric-organ of a plurality of gastric-organs.

In some aspects, the techniques described herein relate to a method, wherein the presentation of the gastric-determined information includes an indication of muscle activity in the plurality of gastric-organs over the time period.

In some aspects, the techniques described herein relate to a method, wherein the plurality of gastric-organs includes at least: small intestines, stomach, and colon.

In some aspects, the techniques described herein relate to a method, wherein the time period is at least one day.

In some aspects, the techniques described herein relate to a method, wherein the time period is multiple days, and the presentation of the cardiac-determined information and gastric-determined information includes at least one of: an indication of daily changes in at least one of the cardiac-determined attributes, and an indication of daily changes in at least one of the gastric-determined information.

In some aspects, the techniques described herein relate to a method, wherein the time period includes a plurality of time intervals, and the cardiac-determined information and the gastric-determined information is obtained for each time interval.

In some aspects, the techniques described herein relate to a method, wherein the time intervals are between about 8 and about 12 minutes.

In some aspects, the techniques described herein relate to a method, wherein a variance of a cardiac-determined attribute is determined over a time duration including multiple time intervals.

In some aspects, the techniques described herein relate to a method, wherein the time duration is one of: one hour, four hours, 8 hours, 12 hours, and 24 hours.

In some aspects, the techniques described herein relate to a method, wherein the time duration is selected based on external events, wherein the external events include one or more of: administration of one or more drugs, change in diet, lifestyle modification, medical procedure, and medical intervention.

In some aspects, the techniques described herein relate to a method including: receiving electromyography (EMG) signals of a gastrointestinal (GI) organ by a cutaneous device; receiving heart rate variability data; identifying rhythmic EMG signals of the GI organ from the received EMG signals, the identifying including: generating a mathematical fit to the received EMG signals; and processing the received EMG signals based on the mathematical fit to identify the rhythmic EMG signals; and determining activity of the GI organ based on the identified rhythmic EMG signals and the received heart rate variability data.

In some aspects, the techniques described herein relate to a method, wherein the heart rate variability data includes a time scale that correlates the heart rate variability data to the rhythmic EMG signals.

In some aspects, the techniques described herein relate to a method, wherein the activity of the GI organ includes an assessment of effects on patient health due to therapeutic decisions.

In some aspects, the techniques described herein relate to a method, wherein the identifying of the rhythmic EMG signals, further includes: identifying simultaneous peaks across a plurality of data channels, the simultaneous peaks being associated with the rhythmic EMG signals.

In some aspects, the techniques described herein relate to a method, wherein the received EMG signals processed by the mathematical fit are between about 1 cycle and about 30 cycles per minute.

In some aspects, the techniques described herein relate to a system including: an electronic device configured to be worn by a user, including: one or more electrode pairs configured to capture electromyography (EMG) signals; one or more sensors configured to measure heart rate signals; a processor communicatively coupled to the one or more electrode pairs and the one or more sensors; and a memory having instructions stored thereon, wherein execution of the instructions by the processor causes the system to: capture EMG signals associated with at least one gastrointestinal (GI) organ of the user; capture heart rate signals of the user; and identify, in the EMG signals or the heart rate signals, rhythmic EMG signals of the at least one GI organ, the identifying including: generating a mathematical fit to the identified EMG signals, and processing the captured EMG signals based on the mathematical fit; and determining activity of the at least one GI organ based on the identified EMG signals and the captured heart rate signals.

In some aspects, the techniques described herein relate to a system, wherein the EMG signals are concurrently captured with the heart rate signals, wherein the heart rate signals indicate an absolute heart rate.

In some aspects, the techniques described herein relate to a system, wherein the processor is further configured to: calculate a long-term heart rate variability (HRV) trend based on one or more time-segmented heart rate measurements; and correlate the HRV trend with the EMG signals to infer a physiological state evinced by the user.

In some aspects, the techniques described herein relate to a system, wherein the processor is further configured to: calculate a heart rate variability HRV metric including a variance of averaged heart rate signals over a time period; and correlate the HRV metric with one or more user-reported symptoms.

In some aspects, the techniques described herein relate to a system, wherein the processor is further configured to: identify a first state of the user based on the heart rate signals; and identify a second state of the user based on the heart rate signals, wherein the processor is further configured to: estimate activity in a predetermined frequency range during the first state, based on the captured EMG signals; estimate activity in the predetermined frequency range during the second state, based on the captured EMG signals; and calculate a ratio of activity power during the first state to activity power during the second state to form a measurement of a relative activity level between the first state and the second state.

In some aspects, the techniques described herein relate to a system, wherein the processor is further configured to: estimate activity in the at least one GI organ during the first state, based on the captured EMG signals; estimate activity in the at least one GI organ during the second state, based on the captured EMG signals; and calculate a ratio of activity power during the first state to activity power during the second state to form a measurement of a relative activity level between the first state and the second state.

### BRIEF DESCRIPTION OF THE DRAWINGS

FIG. 1 illustrates a schematic diagram of one embodiment of a wearable, wireless, GI electrodiagnostic data aggregating and diagnostic system.

FIG. 2 illustrates a schematic diagram of one embodiment of a multi-electrode configuration of a wearable, wireless, GI electrodiagnostic data aggregating and diagnostic system.

FIG. 3 illustrates a schematic diagram of a simplified bottom view of one embodiment of a patch of a wearable, wireless, GI electrodiagnostic data aggregating and diagnostic system.

FIG. 4 illustrates a functional view of the system of FIG. 1 with various circuit modules including a processor and memory to execute software.

FIG. 5A illustrates time series data recorded on a single channel from a subject's abdomen.

FIG. 5B illustrates a Fast Fourier Transform (FFT) spectrum of the same data of FIG. 5A.

FIG. 6A illustrates an example mathematical fit of example spectral data performed for a spectrum.

FIG. 6B illustrates another example mathematical fit of example spectral data performed for a spectrum.

FIG. 6C illustrates another example mathematical fit of example spectral data performed for a spectrum.

FIG. 7 illustrates the ability of the Gaussian fit to accurately identify a baseline value which results in an optimized calculation of the true area under the curve.

FIG. 8A illustrates a spectrum with about three peaks, with two peaks being recognized by the analysis process as being valid.

FIG. 8B illustrates how removal of the two larger peaks by subtraction of the Gaussian mathematical form allows a third peak to be recognized by the analysis process in a second pass through the data.

FIG. 9A illustrates a spectrum depicting two peaks and a broad area of activity above a nominal background.

FIG. 9B illustrates how removal of the two peaks in FIG. 9A reveals the shape of the broad activity, and subsequently after removal of the nominal background.

FIG. 10A illustrates a spectrum with four peaks, two narrow and two broad.

FIG. 10B illustrates the same spectrum as in FIG. 10A but with the analysis using a different set of tuning parameters in a second pass through the data, in this example detecting the broad peaks but not the narrow ones.

FIG. 11A illustrates a spectrum from a data file that has been cleaned to the 50,000 unit level.

FIG. 11B illustrates the same channel and time segment but with the time series data cleaned to the 5,000 unit level.

FIG. 12A illustrates a weighted peak histogram of a full test using the data file cleaned to the 100,000 unit level.

FIG. 12B illustrates the weighted peak histogram of the same test as in FIG. 12A, but using the data file cleaned to the 5,000 unit level.

FIG. 13A is another illustration of a spectrum that does not exhibit clear peaks when cleaned up to the 50,000 unit level.

FIG. 13B illustrates the emergence of a clear, detectable peak when the cleanup level is 10,000 units.

FIG. 14 is a flowchart of an example process for analyzing spectral peaks associated with movement in a gastrointestinal tract.

FIG. 15 is a flowchart of an example process for analyzing spectral peaks associated with movement in a gastrointestinal tract.

FIGS. 16A1-16A4 illustrate a set of weighted and unweighted daily peak histograms.

FIGS. 16B1-16B4 represents FIG. 16A1-16A4 measured using a test where the patient exhibits significant day to day variation in the 3 cycles per minute (cpm) region.

FIG. 17A illustrates a weighted peak histogram constructed from data acquired during typical daytime (waking) hours for a single subject.

FIG. 17B illustrates a weighted peak histogram constructed from data acquired during typical nighttime (sleeping) hours for the same subject as in FIG. 17A.

The illustrated embodiments are merely examples and are not intended to limit the disclosure. The schematics are drawn to illustrate features and concepts and are not necessarily drawn to scale.

### DETAILED DESCRIPTION

Disclosed herein are systems and methods for processing myoelectrical signals of the gastrointestinal tract. In general, gastrointestinal (GI) myoelectrical signals originating in the GI organs (e.g., such as the stomach, small intestine, and colon) can be measured cutaneously. Such measurements of the myoelectrical signals may represent motor activity arising from contractions of one or more muscles in the walls of the GI organs. These contractions may serve to break up, mix, and/or propel the contents of the GI tract/organ(s), which begin a journey through the GI tract as food. The muscle contractions may be controlled by specialized cells known as Interstitial Cells of Cajal (ICC) that set up pacemaker rhythmic electrical control signals. These electrical control signals are low frequency measuring a few cycles to a few tens of cycles per minute (cpm), depending on the organ, the specific location in the case of the small intestine, and the specific function, in the case of the colon. The electrical control signals may have a low amplitude that may be difficult to measure at a skin surface site. Instead, the signals that are detected at a skin surface site may be a result of the bursts of electrical activity released whenever muscle cells contract in the GI organs and/or tract. When under control of the ICC pacemaker, many muscle cells contract synchronously resulting in an electrical signal of sufficient amplitude to be detected at the skin surface, and such signals may also be distinguishable (due to the rhythmicity) from other sources of background noise and/or competing electrical activity. When detected at the skin surface, the signals of the GI tract/organs are on the order of millivolts (mV). As such, surrounding noise may make it difficult to obtain such signals from a skin surface site because competing electronic signals include noise inherent to detection electronics, noise generated at the interface between skin and electrodes, artifacts from movement causing changes in distance between paired electrodes or shifting of electrodes relative to skin, and myoelectric signals from skeletal muscles or the heart may interfere with the GI tract/organ signals.

Furthermore, being intimately connected to muscular contractions, the myoelectric signals from the GI organs may provide information about the motor function of the GI tract or gut, generally referred to as GI motility. In addition, many disorders of the gut are either directly linked to GI motility, or strongly influenced by GI motility. Common examples of such disorders include, but are not limited to constipation, diarrhea, bloating and distention, vomiting and nausea, and/or chronic abdominal pain. Existing conventional GI measurement techniques used to measure motility include antro-duodenal manometry, colonic and colorectal manometry, gastric emptying scintigraphy involving a radioactive meal and images detected by a gamma camera, and Sitzmark studies of swallowed opaque markers with x-rays over several days, all of which have one or more limitations in terms of patient acceptance and accuracy.

The systems and methods described herein provide an advantage of conventional GI measurement techniques by providing an ability to monitor motility in a non-invasive manner using one or more electrodes or electrode patch(es) applied cutaneously and which are configured to capture data at any point in time without having to intake particular bowel preparation or food before measurements and in situations in which a subject (e.g., patient) may be ambulatory and/or otherwise moving. Conventional techniques are configured to function when the subject is in clinic, supinely situated, and/or having ingested special bowel preparations. The systems and methods described herein may provide a remote and wearable system that enables subjects to go about typical daily life during measurements of the GI tract/organs, which may provide an advantage of increasing patient acceptability. The systems and methods described herein may record measurements for multiple days to capture a full range of gut activity, which naturally varies over the course of a day (and between night and day) to provide improved insight over conventional systems into the function and/or disorder of a GI tract and/or GI organs of the subject.

The systems and methods described herein may measure myoelectric signals, determine an amount and/or nature of motor activity in one or more GI organ as a function of time, and/or report such findings to a physician. The systems and methods described herein may use various signal processing techniques to quantify an amount of energy in a given frequency band. For example, the systems and methods described herein calculate a frequency spectra using Fast Fourier Transform in a succession of consecutive or partially overlapping time segments. When rhythmic activity generated by underlying organ motor activity of sufficient strength is detected, a peak will appear in the spectrum at the associated frequency. This myoelectric data of the GI tract acquired from the abdominal skin surface, for example, may be characterized by a combination of relatively high amplitude brief artifacts, broad spectrum, low level random noise, and rhythmic signals at specific frequencies. The artifacts may stem from internal body sources such as skeletal muscle contractions as well as from interactions of the electrodes and skin surface that can be due to motion of one relative to the other. The low level background may have many sources, some of which are related to the GI tract and some from skeletal muscles, while others are related to the sensing hardware and its interactions with the skin surface. Typically, for all but the youngest children the frequency range of interest is in the 1 to 30 cycles per minute (CPM) range, as will be described in further detail herein.

### System

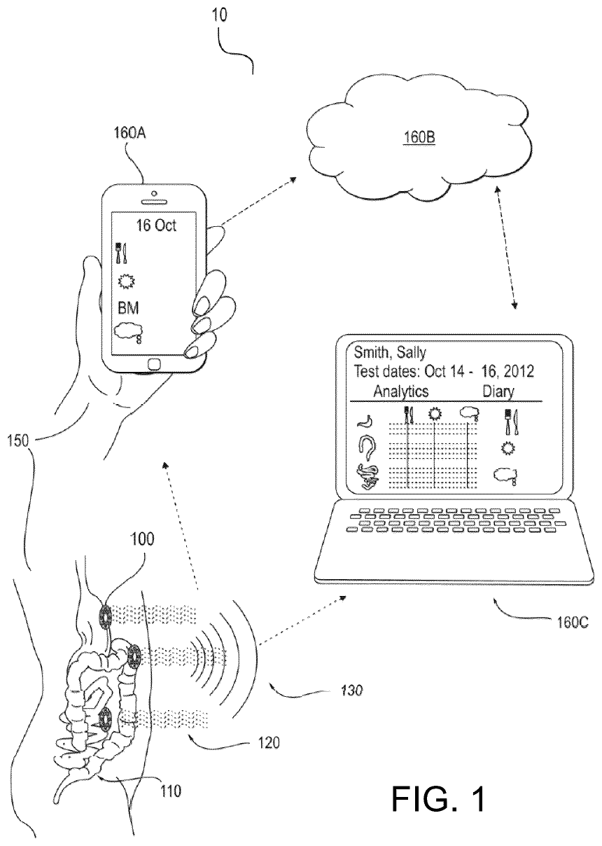

FIG. 1 schematically illustrates an example of a wearable, wireless, GI electrodiagnostic data aggregating and diagnostic system 10. In the depicted embodiment, the system 10 includes one or more patches 100. The patch 100 may provide long-term non-invasive GI tract/organ monitoring. The patch 100 may be an inexpensive, light, water-resistant, and disposable skin-adhesive unit. Because the patch 100 is disposable, it can be easily replaced with another disposable unit after its usage for a period of time (e.g., hours, days, weeks).

The patch 100 may communicate with a mobile device 160A such as a smartphone, wearable device, or other portable computing device, using a low energy protocol 130 such as Bluetooth LE, for example. The system 10 also includes a cloud-based server 160B for storage of data uploaded by the mobile device 160A via standard protocols 130 such as Wi-Fi or cellular phone connections. Processing may occur on the mobile device 160A, in the cloud 160B, or on a separate communicatively coupled computing device 160C, or a combination thereof, to provide information related to the motor activity of the GI tract 110. In some embodiments, the system includes: one or more patches 100 (including one or more electrodes) for acquiring signals from the GI tract 110 of a patient 150, a handheld computing device 160A (e.g., mobile phone, tablet, etc.) for supplemental data entry by a patient or other user, one or more remote computer servers 160B, and one or more remote computer/display devices 160C. In some embodiments, electrical signals 120 of the body may be acquired by the one or more patches 100, patient symptoms and activity may be entered by a user of the handheld computing device 160A, and data indicative of the electrical signals and user-entered information may be wirelessly transmitted 130 to the remote computer servers 160B for data storage and optional processing. The data may be further accessed by, and optionally transmitted to, the remote computer/display device 160C for further processing and/or display. In some embodiments, the processed data is displayed as a table or plot of EMG (electromyography) activity and for doctor diagnostic assistance. In some embodiments, the system includes one or more features of the systems described in U.S. application Ser. No. 14/051,440, titled “Wearable wireless patches containing electrode pair arrays for gastrointestinal electrodiagnostics,” filed Oct. 10, 2013, which is herein incorporated by reference in its entirety.

In some embodiments, the computing device 160A is capable of allowing a subject to enter activity and symptom information relevant to their GI tract such as meal contents, bowel movements, and/or abdominal pain, synchronizing and combining this information with the time-stamped raw data 120 and uploading both to a cloud server 160B or other wireless host. The host may serve as a repository of data. The host 160B may also serve as a processing device for further processing; in such embodiments, the host 160B may perform various methods described herein, and highly processed data is available for download for viewing or further manipulation on one or more remote computing and display devices 160C. Alternatively, relatively unprocessed data may be downloaded to a remote computing and display device 160C for further processing. In such embodiments, the remote computing and display device 160C serves as the processing device configured to perform various processing methods described herein.

Each patch 100 may be a multi-day wearable patch that may sense and digitize myoelectric data 120 at the skin surface of the patient 150 that originated in the smooth muscles of the stomach, small intestine, or colon of the GI tract 110. The patch 100 may transfer the myoelectric data wirelessly 130 to the handheld computing device 160A or another device. In some embodiments, the patches 100 may include two or more bipolar pairs of electrodes 205 arranged substantially orthogonally. The bipolar pairs of electrodes may be configured to sense and acquire EMG voltage signals. Further, the patches 100 may include onboard sensors that are capable of measuring acceleration, velocity, and/or position. The sensors of some embodiments include one or more of accelerometers, GPS sensors, and/or gyroscopes.

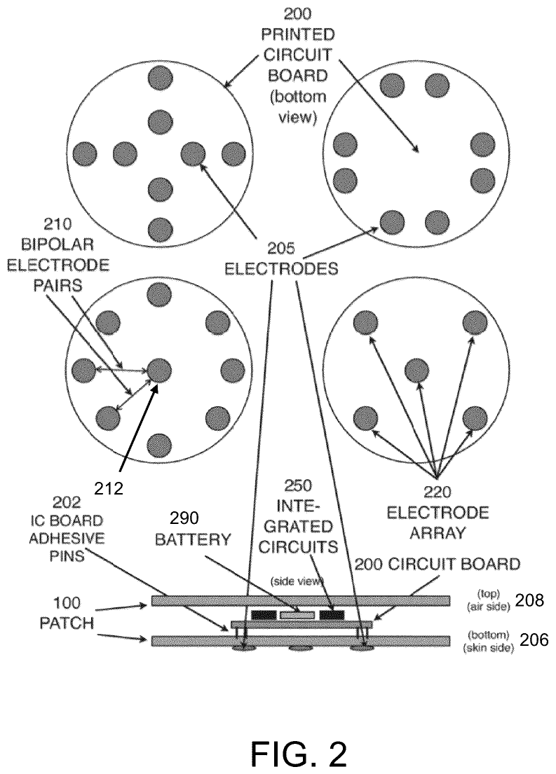

FIG. 2 illustrates a schematic diagram of one embodiment of a multi-electrode patch 100 configuration of a wearable, wireless, GI electrodiagnostic data aggregating and diagnostic system 10. Each patch 100 includes a circuit board 200, which includes integrated circuit components 250 and a battery 290 to supply power to the patch. The integrated circuit components 250 of various embodiments may include a microprocessor, memory, and one or more signal processing components such as an operational amplifier and AC-DC converter, which function together to digitize, amplify, and optionally, filter or otherwise process the acquired voltage signals. The integrated circuit components 250 further include a wireless antenna for modulating and demodulating digitized data for wireless signal transmission. In some embodiments, the wireless antenna is a radiofrequency (RF) antenna configured to wirelessly receive and transmit digital signals to the handheld computing device 160A. In some embodiments, the RF antenna is a Bluetooth®, low energy Bluetooth® antenna, iBeacon, nearfield communication antenna, or any other suitable RF antenna configured to transmission of signals from one or more patch 100 to the handheld computing device 160A.

The computing devices described herein (e.g., processor 422, processor in components 250, etc.) of various embodiments may be part of a sensor device of patch 100, a laptop, a tablet, a desktop computer, a server machine, or other computing device. The computing devices may use time and frequency based algorithms to extract events and patterns of events that relate to the activity of the aforementioned GI organs, specifically slow waves that are associated with mixing and propulsion of their contents as part of digestion and elimination, with the purpose of providing motility data and/or diagnostic information on the activity of the organs as they relate to function or dysfunction of the GI tract/organs. In some embodiments, the computing devices perform artifact removal, normalization, and characterization of the extracted events and patterns that relate to the activity of the GI organs, as will be described in greater detail below. The computing devices further include an ability to coordinate data transfer schedules with any number of patches 100 to accommodate either regularly scheduled transfers of data or reconnecting when temporarily out of range, and the further ability to identify patches individually.

In some embodiments, the remote computing and display device 160C includes: a wireless communication connection to access data from the remote server 160B; and a touchscreen, monitor, LCD screen, or other monitor configured to graphically display the processed data to a physician or other healthcare professional. The processed data of some embodiments is displayed on the remote computing and display device 160C in a line graph, table, or other output format that can be quickly and easily interpreted and understood by a healthcare professional. In some embodiments, the displayed data is used to facilitate diagnosis or monitoring of one or more gastrointestinal function and/or dysfunction (e.g., disorders).

As shown, any embodiment of the patch 100 may include the electrode array circuit board 200. The circuit board 200 may include two to ten embedded bipolar pair electrodes 205. In some embodiments, the circuit board 200 includes at least two embedded bipolar pair electrodes 205. In some embodiments, the circuit board 200 includes eight embedded bipolar pair electrodes 205 arranged in an electrode array 220. In some embodiments, an inter-electrode distance is between about one and about two inches. The electrodes 205 may be embedded inside the printed circuit board 200 of patch 100, with a slight extension for greater skin contact. In some embodiments, the circuit board 200 is entombed in waterproof resin for greater water resistance. Further, in some embodiments, a patch housing surrounding the circuit board 200 may include water resistant properties.

The patch 100 may include a bottom layer 206 (e.g., a skin-side layer) with two or more bipolar electrode pairs 210 positioned on a bottom surface of the bottom layer 206. A plurality of integrated circuit (IC) board adhesive pins 202 extend through the bottom layer 206 to connect each electrode 205 of the bipolar electrode pairs 210 to the integrated circuit board 200 positioned on a top surface of the bottom layer 206. In some embodiments, the electronics of the integrated circuit board 200 are protected from moisture and patient manipulation by being sandwiched between a waterproof top layer 208 (e.g., an air-side layer) and the bottom layer 206. In some embodiments, the circuit board is encased in a watertight inner pouch. In some embodiments, the circuit board is encased in epoxy. The integrated circuit board 200 includes one or more integrated circuits 250 and a battery 290. The integrated circuit board 200 may also include signal processing components. For example, the integrated circuit board 200 may include one or more of: a filter (e.g., low pass filter, high pass filter, or band pass filter), an amplifier, an analog-to-digital converter (ADC), and a processor (or microcontroller) to process and analyze signals received from the electrodes 205. The patch 100 may further include a transmitter or a transceiver antenna to transmit signals from the patch 100 to the cloud-based server 160B or the mobile device 160A, as shown in FIG. 1.

In some embodiments, each patch 100 includes two, three, four, five, six, seven, eight, nine, ten, or more electrodes 205. In some embodiments, the patch 100 includes four bipolar electrode pairs 210. In some such embodiments, the patch 100 also includes a grounding electrode 212, for a total of nine electrodes 205. The ground electrode 212 may be disposed substantially in a center region of patch 100, such that electrodes 205 are disposed circumferentially around ground electrode 205. In some embodiments, ground electrode 212 may be positioned anywhere on patch 100. The electrodes 205 may be electrically coupled via arms (not shown). The arms may be arranged such that patch layer 206 conforms and bends or flexes in two dimensions along two central and perpendicular axes. Further, in some embodiments, a plurality of interchangeably paired electrodes 205 may form the electrode array 220. In some such embodiments in which the electrodes 205 may change the pairings of electrodes during signal acquisition, fewer electrodes may be used to determine directionality, strength, and/or breadth of the signal. While a few example electrode arrangements are shown in FIG. 2, those skilled in the art will appreciate that any suitable electrode arrangement may be used and is contemplated herein. Moreover, while a circular patch is shown, it is also contemplated that the patch may be rectangular, star shaped, oval, or any other suitable regular or irregular shape.

In the systems described herein, using pairs of bipolar electrodes on one or more patches, it is possible to dynamically alter which electrodes form bipolar pairs based on incoming data optimization algorithms and/or alter which patch is used for data analysis. For example, the raw data coming from each patch 100 may be analyzed for signal strength. If a signal strength for a particular patch is above a certain threshold, that patch may be selected for further data processing, as described elsewhere herein. Additionally, some or all channels coming from all patches may be analyzed for signal strength and/or quality, such that a particular channel on a particular patch is selected for further data processing, as described elsewhere herein.

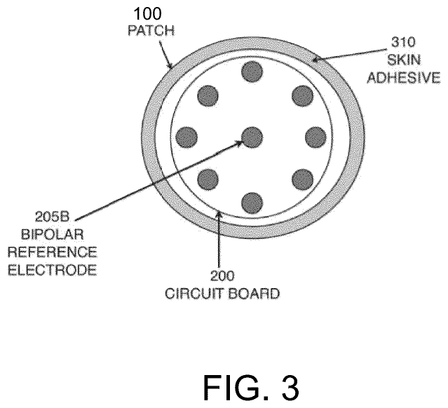

FIG. 3 illustrates a schematic diagram of a simplified bottom view of one embodiment of the patch 100. In some embodiments, the bottom of the patch 100 has an adhesive surface 310 that can be affixed to the skin of a subject for 7-14 days. In some embodiments, the bottom of the patch 100 can be affixed to the skin for at least 7 days. In some embodiments, the adhesive includes a drying adhesive (e.g., white glue, rubber cement, contact adhesives), pressure-sensitive adhesive, contact adhesive (e.g., natural rubber, neoprene), hot adhesive (e.g., hot glue), or multi-part adhesive (e.g., Polyester resin and polyurethane resin, polyols and polyurethane resin, acrylic polymers and polyurethane resins). In some embodiments, the adhesive is a pressure-sensitive adhesive, which forms a bond when pressure is applied to stick the adhesive to the adherent (e.g., the skin).

The patch 100 described herein may acquire myoelectrical data in the form of voltage readings that represent electrical activity of the digestive organs. Inevitably, the electrode patches also sense and record electrical activity from other biological sources, such as the heart and skeletal muscles. Due to the sensitivity of measuring microvolt level signals from the digestive organs, artifacts can be induced by interactions between electrodes and skin surface, for example by way of transverse slippage or partial separation. At least some of these artifacts can be much larger in amplitude than the digestive organ-based signals of interest. Further, these recordings, taken over a period of many hours or even days at frequencies of several Hz or more, and on multiple channels, result in very large data sets, with tens to hundreds of millions of individual readings. Interpreting these data and providing a clinically valuable summary is a significant challenge, which is addressed by the presently disclosed systems using one or more of the methods described herein.

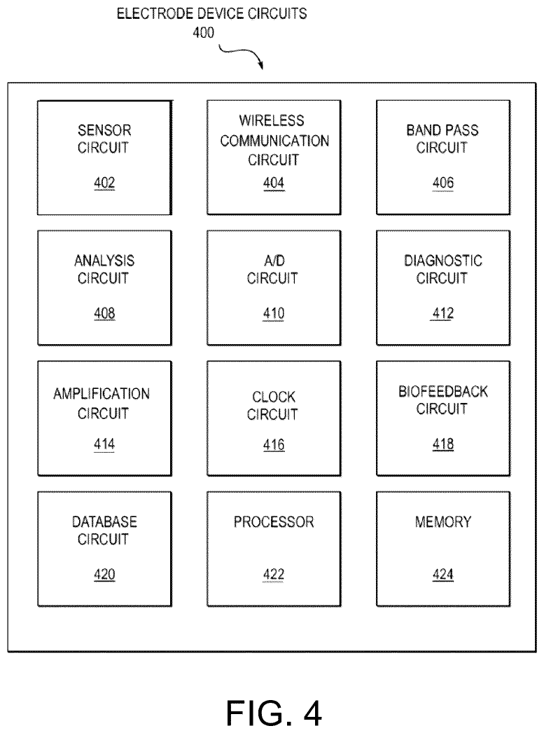

FIG. 4 illustrates a functional view of the system of FIG. 1 with various circuit modules. As shown, an electrode device circuit 400 may include a sensor circuit 402, a wireless communication circuit 404, a band pass circuit 406, an analysis circuit 408, an analog/digital circuit 410, a diagnostic circuit 412, an amplification circuit 414, a clock circuit 416, a biofeedback circuit 418, a database 420, a processor 422 and/or memory 424. The computing devices and/or processors of various embodiments described herein may include or have access to one or more processors 422, which may include one or more microprocessors, digital signal processors (DSP), field programmable gate arrays (FPGA), application specific integrated circuits (ASIC), or other programmable logic devices, or other discrete computer-executable components designed to perform the functions described herein. The computing devices and/or processors may also be formed of a combination of computing devices, for example, a DSP and a microprocessor, a plurality of microprocessors, one or more microprocessors in conjunction with a DSP core, or any other suitable configuration.

In some embodiments, the processors 422 may be coupled, via one or more buses, to memory 424 in order to read information from and write information to the memory 424. The processor 422 may additionally or alternatively contain memory 424. The memory 424 can include, for example, processor cache. The memory 424 may be any suitable computer-readable medium that stores computer-readable instructions for execution by computer-executable components. For example, the computer-readable instructions may be stored on one or a combination of RAM, ROM, flash memory, EEPROM, hard disk drive, solid state drive, or any other suitable device. In various embodiments, the computer-readable instructions include software stored in a non-transitory format. The software, when executed by the processor, causes the processor to perform one or more operations described elsewhere herein.

In operation of system 10 (and device circuit 400), the analysis circuit 408 and/or processor 422 may obtain measurements (e.g., from sensor circuit 402) of the myoelectric signals associated with the GI tract/organs of a subject. Such, measurements of the myoelectric signals may provide data on the motor activity of the GI organs, which can enable understanding the signal output according to a relatively continuous hour to hour and day to day function. The continuous capture of signals from the GI organs may provide a diagnostic aid for those experiencing motility disorders of the gut such as Irritable Bowel Syndrome (IBS), Inflammatory Bowel Disease (IBD), gastroparesis, constipation, diarrhea, or temporary dysfunctions such as post-operative ileus (POI).

Continuously capturing GI tract/organ motility data (i.e., the myoelectric signals from sensor devices of patch 100, for example, may result in artifacts based on subject movements or anomalies occurring to the sensors during capture. Such artifacts may be large amplitude artifacts that are several orders of magnitude larger than the rhythmic signals of interest of the GI tract/organs. The presence of a large artifact may inadvertently mask any signal of interest in a spectrum if not removed prior to a spectral transformation calculation. A background signal may exist in the captured data. The background signal may include a lower magnitude measurement that may extend from the lowest frequencies measurable to well beyond an upper limit of interest (e.g., about 30 cpm). The background signal may have a gradual decrease with frequency. When motor activity of a GI organ is strong, the resulting signal can be many times higher than the background signal, so that the motor activity signal stands out clearly in a spectrum. Weaker signals, at the beginning or end of a period of activity, (or from a lower level of activity e.g., lower amplitude, or from a smaller section of an intestine, or from a subject where signals are reduced due to larger girth or poorer electrode-skin contact) can be on the order of the background signal in amplitude. Although of lower amplitude and/or activity, the existence of such signals can also be of significant clinical interest for assessing the GI tract and/or GI organs.

Spectral peaks that are large relative to a level of a background signal can be detected using various threshold techniques to remove/cut peaks, as described in U.S. application Ser. No. 15/249,695, titled “Apparatus and method for detecting gastrointestinal motor activity during post-operative recovery,” filed Aug. 29, 2016, which is herein incorporated by reference in its entirety. However, in order to provide additional data for the gut activity of a subject, the systems and methods described herein may detect and quantify the characteristics of low amplitude peaks, while avoiding misidentification of random noise or artifacts as being the result of or associated with true rhythmic gut activity.

From a medical diagnostic perspective, recording, identifying and quantifying as much of the rhythmic myoelectric motor activity as possible, whether strong or weak may ensure that peaks in the data are not missed. However, mathematically speaking, the energy of rhythmic activity in time series data corresponds to the area under the curve in a spectrum of such data. The analysis circuit 408 may include algorithms that may extract all (or a portion of) rhythmic signals that are present in the spectral data in order to quantify a level of activity and to discriminate against false, random or accidental signals that become increasingly more prevalent as the threshold for detection is lowered. In some embodiments, the extracting may be performed by another computing device 160A, 160B and/or 160C.

### Gaussian Mathematical Fit

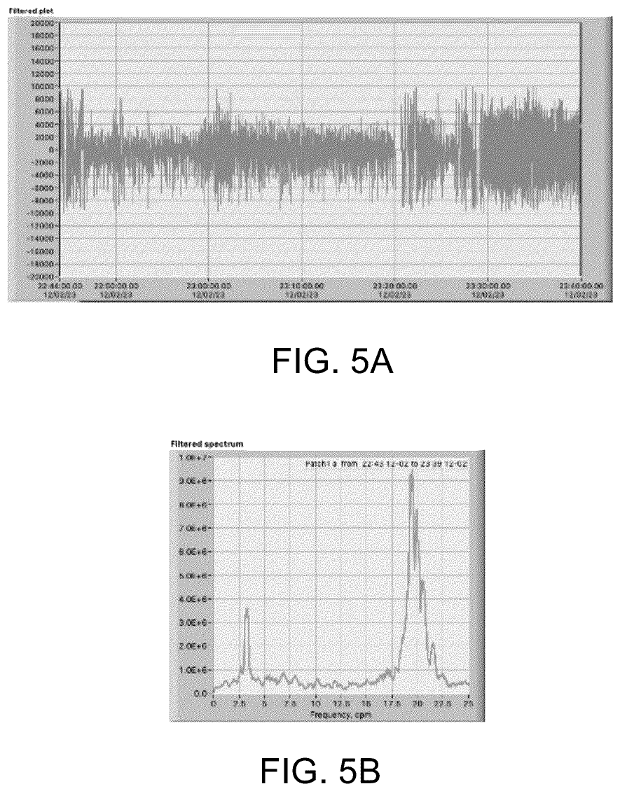

FIG. 5A illustrates time series data recorded on a single channel from a subject's abdomen. FIG. 5B illustrates a Fast Fourier Transform (FFT) spectrum of the same data of FIG. 5A exhibiting clear peaks including a smaller, narrow peak at about 3 cpm and larger and broader peak centered at about 19 cpm. The processor 422 (or another computing device 160A, 160B and/or 160C) may obtain measurements such as the time series data in FIG. 5A and may then determine the spectral data shown in FIG. 5B. The processor 422 (or another computing device 160A, 160B and/or 160C) may then estimate an area above a baseline level of a particular signal. As described herein, the energy of the underlying motor activity of the GI organs may be represented by the area under the curve of the spectral peak, excluding the baseline (or background signal). In order to track the organ activity, shorter time segments may be desirable, as the underlying activity can be as brief as a few seconds to a few minutes. However in fundamental terms, the shorter the time segment, the less information is present.

The width of a frequency peak (in units of cpm) is inversely related to the duration in the segment of time. For example a ten minute segment will have a frequency resolution of about 1/10 cpm. Since the area under the curve is determined by the amount of motor activity, a wide peak will not have as much height as a narrow peak. Identifying and measuring the parameters of a peak that sits on top of background becomes increasingly challenging as the peak height becomes less in comparison to the level of background signal. Unfortunately the nature of cutaneously measured signals from the GI tract is such that the signals of interest are often on a similar order or even smaller than the background level. Therefore, the system 10 provides for a computerized and automated algorithmic approach that can optimize the detection of true peaks while discriminating against random noise peaks, separate signal from background, and quantify peak parameters would of great value in monitoring GI motor activity and ultimately in aiding patient care.

While the techniques described herein may utilize finding data points that are above a specified threshold in order to group contiguous sets of points as a single peak, filtering may be applied to the data to smooth out rapid variations for peaks/signals that repeatedly cross the threshold. After smoothing and/or filtering, such techniques can also apply one or more quality tests, or cuts (i.e., signal portion removal/peak removal) to discriminate true from false peaks. For example, determining valid peaks in spectral data may include assessing the amplitude, the amplitude relative to the baseline, the width, and so forth across the calculable parameters of each peak. In some embodiments, the techniques may break the spectrum up into sub-ranges of frequency that each have fewer peaks and less variation of the baseline, as described in U.S. application Ser. No. 18/489,436, titled “Systems and Methods for processing electromyographic signal of the gastrointestinal tract,” filed Oct. 18, 2023, which is herein incorporated by reference in its entirety.

In general, identification of true peaks (i.e., valid peaks generated by the GI tract and/or GI organs) and discrimination against false peaks arising from noise or artifacts (i.e., peaks, noise, artifacts, and/or background signals not indicating GI tract and/or GI organ data) can be improved by applying a mathematical fit of a function which closely approximates the shape of a peak to the candidate peaks. The system 10 may perform such a mathematical fit on any number of signals obtained from one or more electrodes/patches 100. One example function that mimics/approximates a shape of a peak is the Gaussian function, which has the form has the functional form shown in equation [1]:

$$f ( x ) = {ae}^{\frac{- {( x - b )}^{2}}{2 {c}^{2}}} + d \qquad [ 1 ]$$

where a is the amplitude, b is the central frequency, c is the width of the peak, and d is the height of the baseline. Other shapes may be used to perform the mathematical fit. For example, a functional fit of a Gaussian function, a Lorentzian function, or other substantially bell-shaped function (or equivalent shape) may be used to mathematically fit input data to optimize information held by points of the input data. The mathematically fit data may provide an advantage of improved accuracy of the determined peaks within the data over assessments that include analysis of data point by data point values of spectral data versus frequency data. For example, performing a functional fit of a peak using a substantially bell-shaped function may provide new quality parameters (e.g. goodness of fit) which can be subjected to further candidate peak removal as a means of rejecting false peaks. In addition, performing a functional fit of a peak using a substantially bell-shaped function may allow for generating an improved estimation of the parameters of interest, including, but not limited to peak height, peak width, and/or baseline level and area under the curve of the peak.

In addition, the processor 422 (or another computing device 160A, 160B and/or 160C) may combine techniques to assess peaks and other parameters. For example, when a candidate peak has been identified by a preliminary technique, such as the threshold plus peak removal (e.g., cuts) approach, a range in frequencies may be selected on either side of each respective candidate peak and used for the mathematical fitting process described herein. For example, the processor 422 may assess a peak at 3 cpm by using a range of 1 to 5 cpm for the mathematical fitting process.

In some embodiments, particular inputs may be used to guide the mathematical fitting to ensure successfully isolating a valid peak. Such parameters may include, but are not limited to comparison parameters such as frequencies and peak heights of the Gaussian fit and the input threshold based peak. If a mathematical fit fails, a peak may be discarded and identified as not including acceptable characteristics for GI tract/organ signals. If the mathematical fit succeeds, an output from the fit may include a shape function and several parameters, including one or more of: a peak frequency, a peak height above background, a ratio of peak height to background, a peak width as measured by the FWHM (full width half maximum), and a measure of the residual difference between the Gaussian shape function and the input spectrum as absolute parameters.

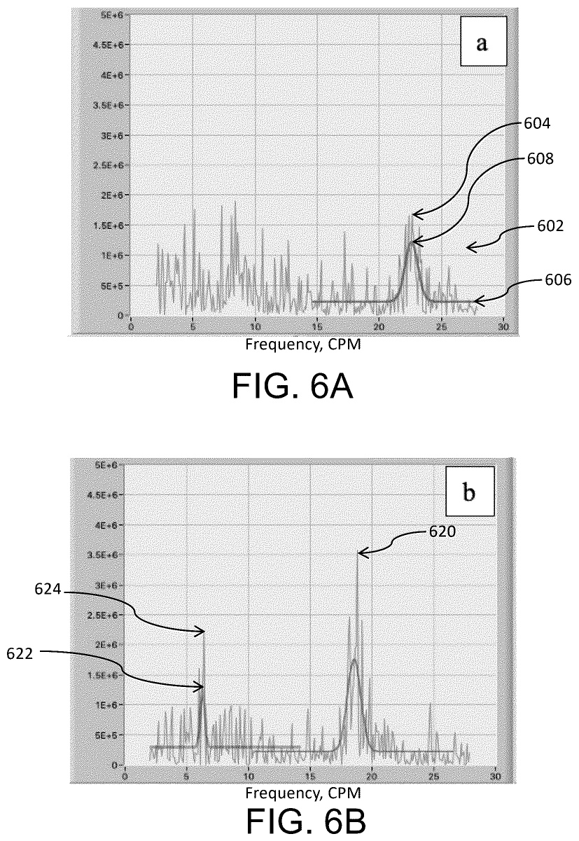

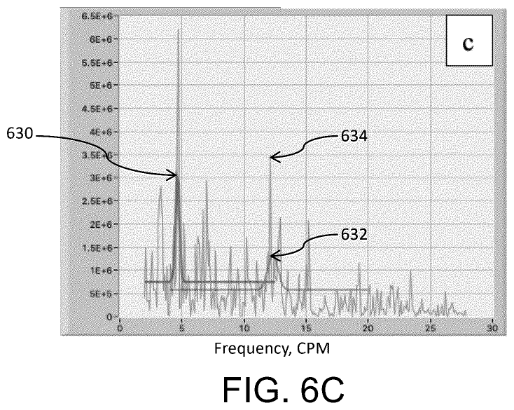

FIG. 6A illustrates an example mathematical fit of example spectral data performed for a spectra capturing about 10 minutes of measurements. Performing/applying a mathematical fit of spectral data derived from electromyographic data of the GI tract, such as time series data acquired from cutaneous patches, to a Gaussian or similar peak-oriented functional shape (or multiple such functions) as a means of determining the existence and measuring quantitative parameters of spectral peaks, reflecting underlying rhythmic activity of one or more of the primary GI organs, the stomach, small intestine and colon. Using these mathematical fit(s) to evaluate the parameters (e.g., center frequency, baseline value, peak width, and the amplitude, and area above the baseline), one or more candidate peaks may be identified and used to track activity of the GI tract during specific time intervals, such intervals ranging from a few minutes to multiple days in length (e.g., about two minutes to about 6 days).

In this example, a Gaussian mathematical fit is performed on data 602 for a peak 604, as shown by line 606 with determined peak 608. For example, the processor 422 (or another computing device 160A, 160B and/or 160C) may receive or determine spectral data from electromyographic data originating from smooth muscles associated with one or more organs of the GI tract using patch 100. A mathematical fit may be executed on the spectral data based on at least one shaping function (e.g., the Gaussian shaping function in the example of FIG. 6A). In some embodiments, executing the mathematical fit includes the use of threshold detection as input to the Gaussian fit. For example, the processor 422 (or another computing device 160A, 160B and/or 160C) may use a traditional threshold based method, across the full frequency ranges or in one or more sub-ranges of frequencies, to identify the approximate frequencies, and/or widths and/or amplitudes of candidate peaks to act as inputs to the fitting algorithm to enhance accuracy and efficiency of the fitting program. Such input may improve the probability of making a fit to an actual peak in the spectral data.

In operation, the processor 422 (or another computing device 160A, 160B and/or 160C) may set (e.g., assign, identify, define, etc.), for the spectral data, a threshold applicable to identifying an approximate amplitude, a range applicable to identifying a width of the one or more candidate peaks and set, for the spectral data, a target value applicable for identifying an approximate frequency of the one or more candidate peaks. The processor 422 (or another computing device 160A, 160B and/or 160C) may use the identified approximate amplitude or approximate width and/or the approximate frequency as input into the mathematical fit to identify the one or more candidate peaks. In some embodiments, the amplitude threshold, the width range and the target frequency are determined based on values within the frequency spectrum associated with the spectral data.

Next, the processor 422 (or another computing device 160A, 160B and/or 160C) may determine, for one or more candidate peaks, a plurality of parameters that quantify underlying rhythmic activity of one or more GI organ of the GI tract and may select, based on the determined plurality of parameters and the mathematical fit, at least one of the one or more candidate peaks to represent activity of the GI tract during a predefined time interval.

A successful mathematical fit of the signal is shown as line 606, which has a peak around 22 cpm. The line 606 has a strong fidelity of a fit to the underlying data 602 during the range from about 14 to 27. No other sections before about 14 and after about 27 in the spectrum satisfied the conditions for a Gaussian fit. This fidelity ensures that the mathematical parameterization provides an increased likelihood of a match to the actual shape of the spectral signal and therefore, a valid quantification of peak parameters.

FIG. 6B illustrates another example mathematical fit of example spectral data performed for a spectra capturing about 10 minutes of measurements. In this example, a successful curve (e.g., mathematical) fit is depicted at about 18 cpm at peak 620 and a failed curve fit is depicted at about 6 cpm at peak 622. The failure in this case may be due to a peak height (i.e., amplitude) at peak 622 being less than a minimum acceptable value associated with the signal peak 624.

FIG. 6C illustrates another example mathematical fit of example spectral data performed for a spectra capturing about 10 minutes of measurements. In this example, a successful curve (e.g., mathematical) fit is depicted at about 5 cpm at peak 630 a failed fit is depicted at about 12 cpm at peak 632. The failure in this case may be due to a high value of residual difference between fit (peak 632) and data (e.g., peak 634), which, like all cuts, may be used to reject likely false peaks.

Each Gaussian fit may result in values of peak height, width, center frequency, and baseline. Determining a reliable baseline value may ensure that any calculation for a parameter related to strength or motor activity (i.e., the area under the peak excluding the baseline) is as accurate as possible. Even a small error in baseline estimation will translate to larger errors in area as the peak is widest at its base.

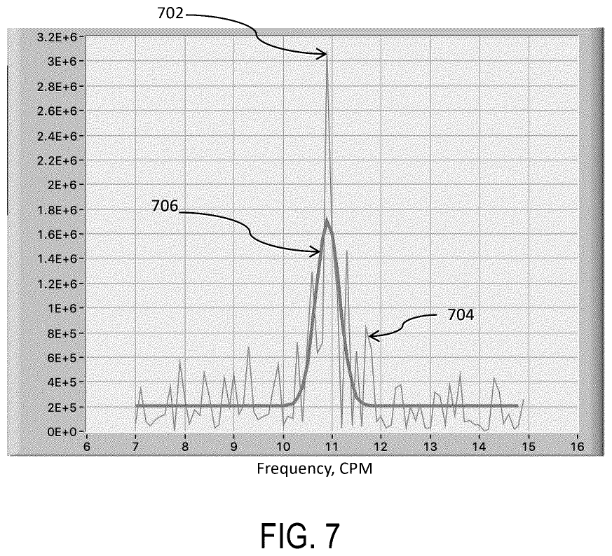

FIG. 7 illustrates the ability of the Gaussian fit to accurately identify a baseline value which results in an optimized calculation of the true area under the curve. The spectral data with a Gaussian fit curve shows how the fitted baseline is well above zero at 2e5, at about 15% of the true peak height of 15e5. The Gaussian fit provides the baseline value as a parameter that has been optimized by least-squares minimization to be a best estimate of the true baseline height. Alternative methods of estimating the baseline exist, such as averaging points outside the peak area for some distance on either side of the peak, but all involve more complicated algorithms, fine tuning of the control parameters, and can be error prone for example if another peak is nearby.

Once a first peak in a given range and time segment is found and verified, the peak can be removed by subtraction of the parameterized formula from the spectrum. For example, the processor 422 (or another computing device 160A, 160B and/or 160C) may obtain measurements for ranges, identify peaks (e.g., peak 702, peak 704, etc.), and may mathematically subtract the fit peak of line 706 from the spectrum using the Gaussian function formula with the parameters determined from the fit upon verification completion. In the example of one or more large peaks and one or more considerably smaller peaks, removal of the larger peaks can improve the detection capability of the smaller peaks, by allowing the system 10 to use lower thresholds if a threshold detection step precedes Gaussian fitting (shown by line 706), or by clearing the field for the minimization routine of the fitting program to converge on the smaller peak.

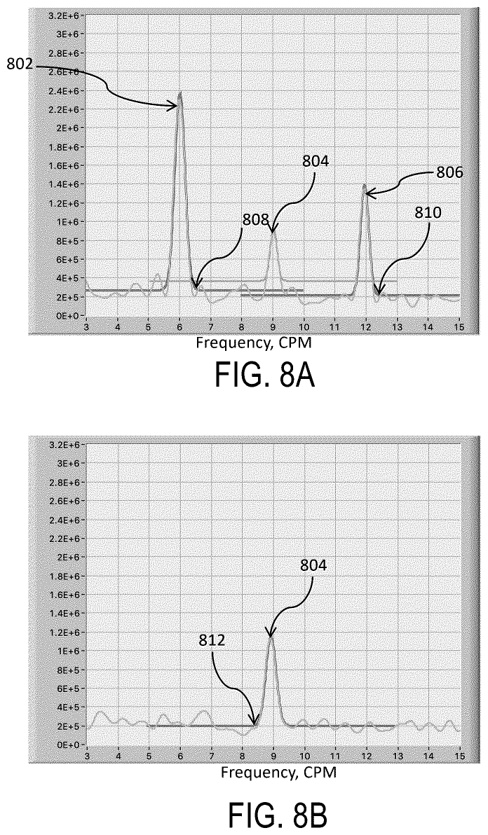

FIG. 8A illustrates a spectrum with about three peaks (e.g., peak 802, peak 804, and peak 806), with two of the peaks being recognized by the analysis process as being valid. The two peaks 802 and 806 depict a successful Gaussian fit graphed by line 808 and line 810, labeled as the fit line rises toward the peaks and mirrors the peaks 802, 806, respectively. Peak 804 is present in the spectral data, but has failed the election process due to the influence of the larger peaks 802, 806 causing peak 804 to have a higher baseline in the fit and therefore peak 804 does not match the actual data adequately according to predefined data conditions.

FIG. 8B illustrates how removal of the two larger peaks 802, 806 of FIG. 8A by subtraction of the Gaussian mathematical form to allow the third peak 804 to be recognized by the analysis process in a second pass through the data. Since the larger peaks 802, 806 have been removed, the process of determining peaks may assess peak 804 and perform a fit and quality test (e.g., see Gaussian fit line 812, which rises from the label and mirrors the peak 804), as described elsewhere herein.

This process of removing (e.g., cutting) confirmed peaks can be continued until none remain, leaving a background spectrum. The background shape may be simple, for example linear or monotonically decreasing with low curvature, and therefore consistent with a true baseline that would obtain in the absence of any rhythmic signals from the GI organs, such as would be measured on a part of the body far from the abdomen. In this case a comparison of the average, in ratio to a previously established standard value over the same frequency range, can be used as a normalization standard which can be applied to the area of the peaks, as a correction factor to compensate for such variables as patient girth, skin condition, and any others that may influence transmission of electrical signal strength from muscles of the gut to the electrodes.

In some embodiments, the processor 422 (or another computing device 160A, 160B and/or 160C) may apply filters to spectrum data for either or both threshold techniques and mathematical fit techniques. Each technique may be independently optimized based on such filtering. Providing smoothing filters to the spectral data to address the natural high point to point variation in spectral calculations such as Fast Fourier Transforms, which provide one data point for every two time series points with a very high variance, the smoothing parameters chosen so as to substantially match the expected natural rhythmic characteristics of the underlying gastrointestinal activity and that of its measurement process. Allowing for the filter parameters to be optimized separately for the threshold based detection step, and the mathematical fit based processing using a first threshold applicable to identifying an approximate amplitude or width of the one or more candidate peaks and a second threshold applicable for identifying an approximate frequency of the one or more candidate peaks. The analysis circuit 408 may identify one or more candidate peaks in the spectral data by identifying points within the spectral data that are above the second threshold by executing one or more of a peak detector that imposes constraints including consecutive values above the first threshold, or a peak detector that provides smoothing prior to identifying points within the spectral data that are above the second threshold.

Alternatively the peak-subtracted background spectrum may not be simple, containing residual broad peak-like enhancements that did not meet the cuts of verified peaks. These may be physiologically meaningful and associated with specific organs, in particular the small or large intestines, which have such characteristics in which the active frequencies may shift on time scales shorter than the time window for the analysis. For example, in a small intestine, migrating motor complex as the peristaltic wave progresses down its length, the active frequency, which is tied to location, continually drops. Since the frequency is changing during the time segment its energy will be spread out and not form the type of narrow peak that lends itself to easy detection. In this example, which can be assessed as part of an automated algorithm by evaluation of first and second derivatives (slope and curvature, respectively) which change sign, the residual enhanced frequencies can be quantified as to frequency range and amplitude by subtraction of a simple background shape matched in amplitude at strategic points such as the range limits.

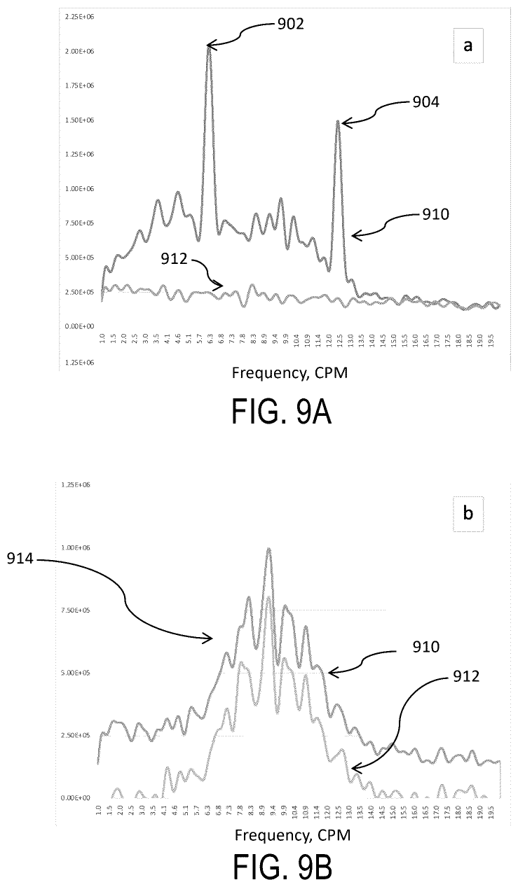

FIG. 9A illustrates a spectrum depicting two narrow peaks (e.g., peak 902 and peak 904) and a broad area of activity above a nominal background. The spectrum also depicts the nominal background with a shape aligned to edges of a first spectrum.

FIG. 9B illustrates how removal of the two peaks (e.g., peak 902, peak 904) in FIG. 9A reveals the shape of the broad activity, and subsequently after removal of the nominal background. In particular, FIG. 9B shows the first spectrum 910 with the two narrow peaks removed using their Gaussian fit parameters, leaving a broad hump 914 sitting atop the background; the second spectrum 912 in FIG. 9B shows the broad hump 914 minus the background. The area under the curve of the broad hump 914 may be used to represent GI organ motor activity which is not steady enough to resolve into narrow peaks, and would otherwise be undetectable if peak detection techniques are used to measure motor activity. In this example, the motor activity represented by the broad area is isolated and therefore may be amenable to being measured and included in reported activity of an organ.

An example of such an algorithm may subtract a peak to increase the ease for the system 1 to find smaller peaks within a particular spectrum. For example, a full range or a sub-range may be used for a first mathematical fit using the largest of the predetermined threshold peaks, after which the functional form from the fit equation may be subtracted from the initial spectrum, leaving a less complex and therefore simpler spectrum to be fit to find the second peak. Each step of the fit may end with the output being removed from the original fit such that another fit is performed to find additional peaks in the spectral data.

In some embodiments, subtraction of peaks may result in a final signal that includes background signal. The background signal may be quantified for ratios or for use as normalization factor. For example, when all peaks for a given frequency range of a spectrum have been fitted and the functional form used to remove the peak from the spectrum by subtraction, the resulting signal for the spectrum is pure background. The average value of the determined background signal may be used in ratio to an established average background level over the same frequency range to function as a normalization standard which can be applied to the area of the peaks, as a correction factor to compensate for such variables as patient girth, skin condition, and any others that influence transmission of electrical signal strength from muscles of the gut to the electrodes.

In some embodiments, one or more peaks may be subtracted to find a residual level above a separately determined background. For example, similar to the above example, each successively found candidate peak may be subtracted from the spectral data leaving the determined background signal. A true background signal (e.g., level) may be determined from the spectral data based on a model associated with a normalization process applied to result in a non-peak based measure of activity within the spectral data. The determined background signal may be compared to the true background signal to result in a residual spectral shape to be used as an additional measure of motor activity in a specific frequency range or subrange associated with one or more organs (e.g., small intestine, large intestine, etc.) which have physiological characteristics wherein the active frequencies may shift on time scales shorter than a time window for the analysis. For example, in a small intestine, migrating motor complex as the peristaltic wave progresses down its length, the active frequency, which is tied to location, continually drops.

In some embodiments, the processor 422 (or another computing device 160A, 160B and/or 160C) may apply cuts (e.g., remove signals) based on one or more fit parameters (e.g., Gaussian fit parameters). For example, following a fitting process, the processor 422 (or another computing device 160A, 160B and/or 160C) may apply pass/fail criteria cuts to remove candidate (e.g., Gaussian) fitted peaks. The criteria may include assessment of the goodness of fit as determined by, for instance, the residual errors between fit and data (e.g., determined via summed, averaged, or weighted average over the frequency range of the fit), agreement between the fit frequency, and/or width and amplitude as compared to the threshold based candidate values. The net effect of the cuts may result in a set of peaks that represent signal examples which have a high likelihood of representing valid underlying rhythmic GI activity. The cuts distributed among multiple parameters may include, but are not limited to amplitude so as not to introduce a bias toward only the strongest of such activity, thereby providing enhanced sensitivity of the system to motor activity of the GI tract where peaks of lower amplitude may play be of interest.

In some embodiments, the fit (e.g., Gaussian fit) may provide an accurate baseline in which to utilize for additional signal analysis. For example, if the area under a curve of a peak is accurately identified using one or more of the mathematical fits described herein, then the energy or activity level of the myoelectric activity that caused the peak may be relied upon to represent actual gastrointestinal activity. To obtain such an accuracy from a fitted curve, the background signal is typically subtracted. However, in a noisy data environment intrinsic to FFT spectra, determining the background signal accurately may be challenging. By using a mathematical fit (e.g., such as a Gaussian peak fit) to then fit to an optimized local background may provide an increase in a likelihood of the background signal being accurately determined. Thus, in some embodiments, executing the mathematical fit of the spectral data includes generating, for the spectral data, an optimized background signal associated with the one or more candidate peaks. Using an optimized background signal in calculations and/or additional data fits may result in an increase in accuracy of the spectral data in the time interval.

The strength of actual signals of interest may exist on a continuum, beginning at barely detectable signals/signal portions due to low amplitude and/or high noise, up to very strong signals/signal portions where baseline is virtually negligible. Even with the advantage of a Gaussian fit to improve the peak detection and selection process makes it can be difficult to simultaneously obtain all peaks (good sensitivity) and reject all random events (good selectivity).

Additional selection criteria that go beyond the signal properties of a single channel of data may aid the effort to simultaneously achieve high selectivity and sensitivity. The orthogonal alignment of the four sets of electrode pairs on the patches (e.g., patch 100) described herein may provide the opportunity to further suppress peaks that result from accidental variations or random noise. For example, the system 10 may use two or more data channels to detect a peak in the same time segment at nominally the same frequency. This process may substantially reduce the likelihood of false or accidental peaks as they are unlikely to happen on separate channels at the same frequency and the same time. Further consistency checks that could be imposed include agreement of the peak widths amongst one or more channels, and the use of temporal information to correlate previous and next time segments. The channels used for comparison may be implanted on the same patch 100, in specific parallel orientations, or simply be any two or more channels available in a test setup.

The orientation of consistent peaks across channels may provide information on the relative location of origin of the internal signal. Signals from above or below the patch 100, for example, may manifest as pairing on vertically oriented electrode pairs, while the horizontal pairs might have consistent signals when the internal source is to the left or right. Signals that appear simultaneously on one or more vertical and one or more horizontal channels may indicate an origin at some angle between vertical and horizontal, which may be inferred by the relative strengths of the measured signals.

The use of two, three, or more simultaneous peaks across the multiple data channels represents a sliding scale of discrimination similar to analog value cuts such as amplitude or width against false peaks. This approach is complementary to the single channel cuts in that it allows discrimination in terms related to spatial extent of the source signal rather than relying on the strength of the signal at a single channel. Complementary analyses can be performed where the data is analyzed in multiple passes with different levels of relative discrimination between single and multi-channel cuts. This may lead to further unique insights into the underlying gut behavior.

Selection of input parameters may target specific features in the data but at the exclusion of potentially meaningful signals that have different characteristics. Similarly, the choice of a particular sub-range of frequencies can result in missed activity or increase the chances of false activity being recorded. These limitations can be overcome by making multiple passes through the data with different settings, and combining the results into an optimized output on a channel by channel and time segment by segment basis. A single instance of each peak per frequency range may be selected from the multiple passes. The selection criteria may be based on peak height, peak area, or any other desirable characteristic. In this approach, the final optimized results may provide a more complete and/or robust analysis than with any single set of control parameters. For example, the methods described herein may be performed iteratively using any variety of different fitting techniques at each step to identify candidate peaks having differing widths.

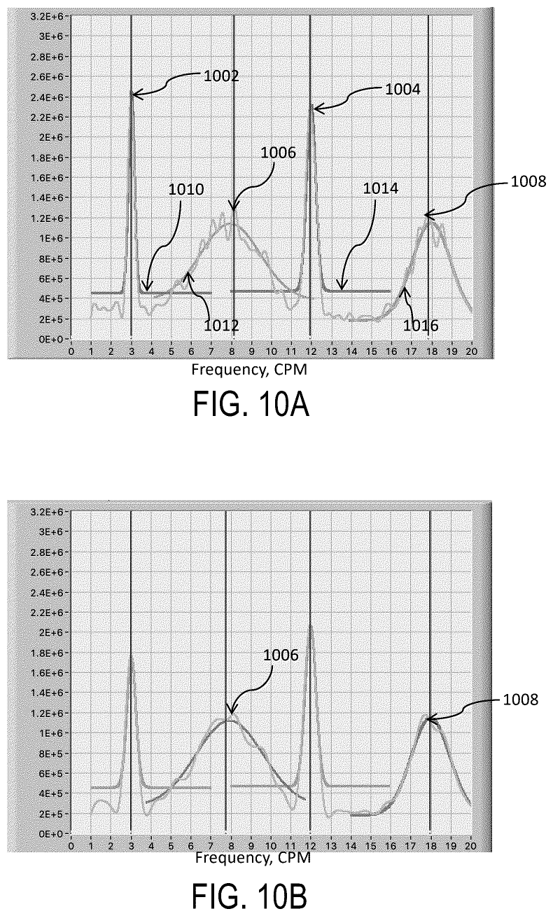

FIG. 10A illustrates a spectrum with four peaks, two narrow (e.g., peak 1002, peak 1004) and two broad (e.g. peak 1006 and peak 1008). On a first cleanup pass, for example, the peak detection and selection parameters may find and verify the two narrow peaks (peak 1002, peak 1004), but the broad peaks (peak 1006, peak 1008) may be rejected due to the tuning parameters, such as degree of smoothing filtering and expected peak width. A respective Gaussian fit 1010, fit 1012, fit 1014, and fit 1016 was found for each respective peak 1002, peak 1004, peak 1006, peak 1008.

FIG. 10B illustrates the same spectrum as in FIG. 10A but with the analysis using a different set of tuning parameters in a second pass through the data. In this example, the second pass may detect the broad peaks 1006, 1008, but not the narrow peaks 1002, 1004. For example, tuning parameters may be set to look for broader peaks and reject narrower peaks. A final meta-analysis of the multiple peak detection passes may allow for all peaks to be included in the final analysis.

### Multiple Cleanup Levels

In some embodiments, the system 10 described herein may employ multiple cleanup levels to find an optimized signal result. For example, the system 10 may preprocess time series myoelectric data to remove artifacts independently at several different cleanup levels, producing a set of input data files corresponding to the different cleanup levels, as a means of revealing peaks of lower amplitude in the presence of either random noise or large peaks in the same time interval, while not attenuating those peaks which have higher amplitudes.

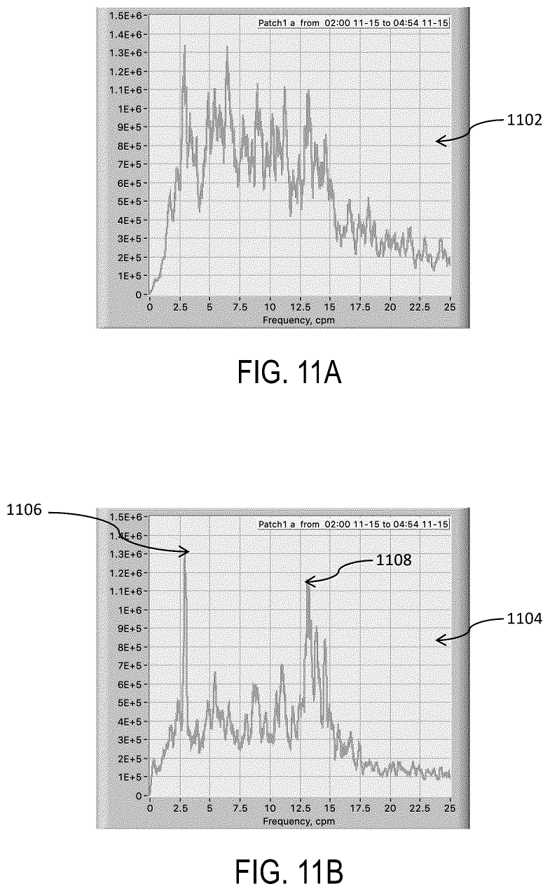

FIG. 11A illustrates a cleaned spectrum 1102 from a data file that has been cleaned to the 50,000 unit level, where no detectable peaks are visible. For example, although relative peaks and valleys of the data are shown, there are no outlier peaks with respect to the remaining spectrum of data.

FIG. 11B illustrates the same channel and time segment but with a cleaned spectrum 1104 of time series data cleaned to the 5,000 unit level, revealing the presence of peaks at about 3 (e.g., peak 1106) and about 14 cpm (e.g., peak 1108).

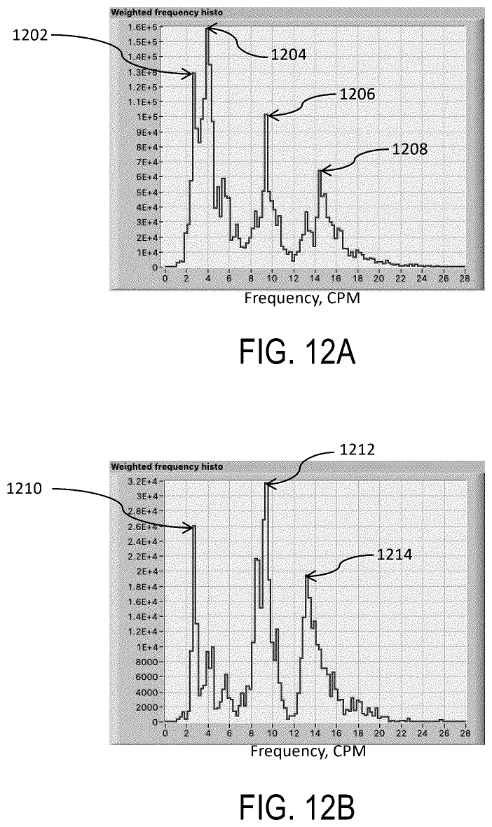

FIG. 12A illustrates a weighted peak histogram of a full test using a data file cleaned to the 100,000 unit level, with clusters of peaks at about 3, about 4, about 9 and about 14 cpm. For example, a peak 1202, a peak 1204, a peak 1206, and a peak 1208 are detected at the 100,000 unit cleanup level.

FIG. 12B illustrates the weighted peak histogram of the same test as FIG. 12A, but using the data file cleaned to the 5,000 unit level. In this example, the balance of histogram contents shifted to different frequencies than in FIG. 12A to show how the different cleanup levels provide complementary information. The relative intensities of peaks at different frequencies are shifted, showing how the various cleanup levels contribute complementary information (e.g., peak 1210, peak 1212, and peak 1214) to the test results.

Processing for peak detection may be performed on each of the multiple cleanup level data files in turn, and the detected peaks (e.g., candidate peaks) in each time interval and on each channel across all cleanup files may be selected, based on an amplitude measurement among all peaks of essentially the same frequency in that time interval and channel. For example, the system 10 may select a peak with a highest amplitude amongst all peaks in the same frequency, time interval, and channel. An additional assessment may be performed by system 10 to ensure that a given peak will appear in more than one file to further reduce the number of accidental peaks.

In general, the presence of high amplitude artifacts in time series data has a strong effect on the spectrum, making it difficult to detect the rhythmic peaks related to GI motor activity. The system 10 may implement the technique for removal of said artifacts using a cleanup level parameter, which sets a maximum amplitude of any data point in the time series. Similar to the effect of obvious high amplitude artifacts, the presence of true rhythmic signals that are particularly strong will also mask the presence of lower strength signal peaks in the spectrum. Further cleanup to lower cleanup levels can reveal these lower level signal peaks, albeit at the expense of losing or at least diminishing the stronger ones. Since it is desirable to include all peaks in the final analysis, the system 10 may process the spectral data (or other associated data set) multiple times. Each processing may occur at a different cleanup level. For example, the cleanup levels may be 100 k, 50 k, 35 k, 20 k, 10 k and 5 k in units of the raw data, representing analog-to-digital converter (ADC) counts, where each count representing 9.3 nano Volts. The peak results may be saved for each run and at the end of this phase of processing, the peak results may be compared in each time segment and in each data channel, across the result files. All peaks found across the result files may be combined in an optimized result file. Typically a given peak frequency will appear in multiple cleanup result files at essentially the same frequency but at different peak heights or areas, in which case a best peak will be selected for the optimized file. The best peak may be the one with the largest peak height, largest area, or some other parameter such as narrowest width or lowest Gaussian residual.

When normalization is applied as compensation between patients for differences in signal strength caused by patient girth, skin condition, or other reason, the reduction in spectral energy when the cleanup level is at its lowest points (which allow certain frequency peaks to emerge that would be otherwise undetected), a bias is introduced because the spectrum in the normalization region of the spectrum will also be suppressed. Since normalization uses the average value of the spectrum compared to a standard value, the normalization may result in an over-correction artificially inflating the strength of any peaks detected. The solution to this problem is to choose one level of cleanup, preferably in the middle of the range, e.g. 35 k, and use the segment by segment and channel by channel normalization factors obtained from it in processing all the files. This can be accomplished, for example, by running the selected file first and storing the normalization values for use in subsequent files rather than recalculating them.

Processing the data across multiple cleanup levels (where it is expected that the same peak will appear in multiple such files) allows for an additional cut to be made to further improve the likelihood that a peak represents valid gastrointestinal data rather than noise. For example, the system 10 may analyze the data output from each cleanup to ensure that any peak that is selected as valid appears in two or more files in order to be included in the optimized result file. The results from each cleanup level may provide value beyond their contribution to the optimized result. When both strong and weak peaks exist simultaneously, or when the noise level is such that the smallest peak are not detectable at cleanup levels, but do emerge at the lower cleanup levels, their contribution to the total motor activity in the optimized file is minimal. Yet the fact of their existence carries physiological significance. For example in gastroparesis patients, one possible cause in a given patient is a lack of ICC (Interstitial Cells of Cajal) which provide the electrical pacemaker signal that stimulates muscular contractions. This state is generally determined by biopsy, which is highly invasive and rarely done. The system 10 may analyze the spectral peaks associated with movement in the GI tract to determine if such signals are detected at about 3 cpm frequency in which these cells produce. If the signals are detected at about 3 cpm, then the existence of the cells is confirmed. Practically speaking this means that a gastric motility stimulant has something to work with and therefore might be effective, in contrast to the case where the ICC are substantially diminished.

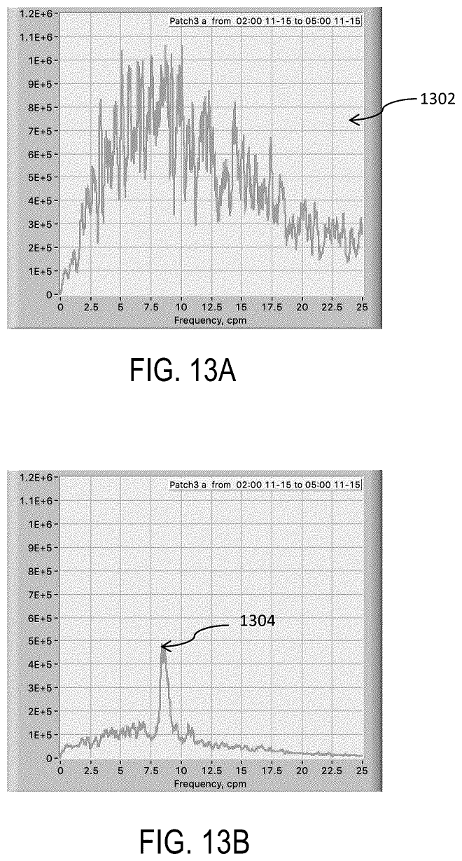

FIG. 13A is another illustration of a spectrum 1302 that does not exhibit clear peaks when cleaned up to the 50,000 unit level. For example, although relative peaks and valleys of the data are shown, there are no outlier peaks with respect to the remaining spectrum of data.

FIG. 13B illustrates the emergence of a clear, detectable peak 1304 when the cleanup level is 10,000 units. For example, the peak 1304 emerged at about 9 cpm due to the reduction in random noise in the data set. This peak 1304 may have a small amplitude relative to other peaks, but it indicates the presence of Interstitial Cells of Cajal (ICC) cells. For example, the metrics of this example provide evidence for the existence of particular cells in the GI tract that function as pacemaker cells in the relevant GI organ even if the net motor activity is at a low level.

### Mathematical Fits

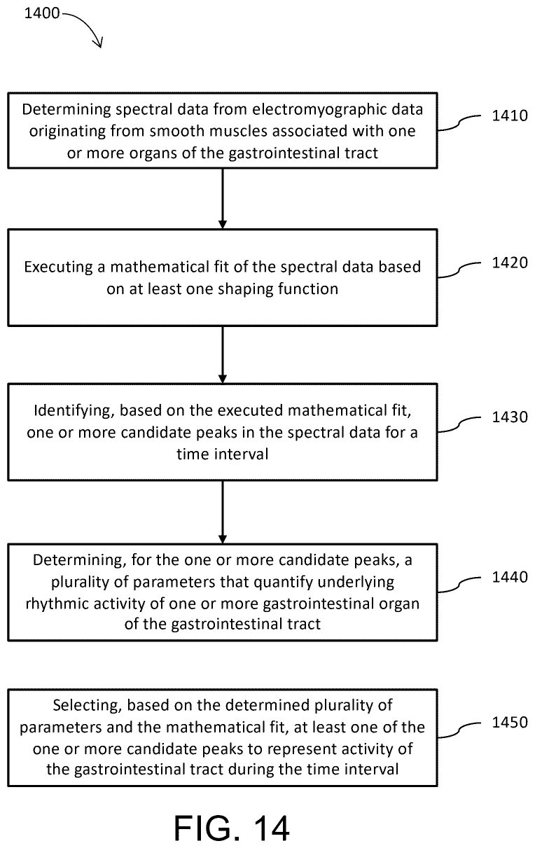

FIG. 14 is a flowchart of an example process 1400 for analyzing spectral peaks associated with movement in a GI tract. The process 1400 may be performed by one or more of the device 400, system 10, and/or other computing device 160A, 160B and/or 160C. In some embodiments, the process 1400 is performed on computing device 160A, 160B, or 160C using data captured by sensor circuit 402, and/or other circuit of device 400. In some embodiments, the process 1400 is performed on processor 422 of device 400.

At step 1410, the process 1400 includes determining spectral data from electromyographic data originating from smooth muscles associated with one or more organs of the GI tract. The spectral data may represent time series data obtained from one or more cutaneous patches (e.g., patch 100) placed on an abdominal region of a subject. The patch 100 may include two or more electrodes, as described elsewhere herein. The one or more GI organs may include at least one of: a stomach, a small intestine, and a colon.

At step 1420, the process 1400 includes executing a mathematical fit of the spectral data based on at least one shaping function. For example, the processor 422 (other computing device 160A, 160B and/or 160C) may include generating, for the spectral data, an optimized background signal associated with the one or more candidate peaks, as described elsewhere herein. The optimized background signal may result in noise reduction of the spectral data in a predetermined time interval.

In some embodiments, executing a mathematical fit of the spectral data based on at least one shaping function includes selecting a shaping function such as a Gaussian function, a Lorentzian function, or other substantially bell-shaped function. In some embodiments, executing the mathematical fit of the spectral data includes setting, for the spectral data, a first threshold applicable to identifying an approximate amplitude or width of the one or more candidate peaks, setting, for the spectral data, a second threshold applicable for identifying an approximate frequency of the one or more candidate peaks, and using the identified approximate amplitude or approximate width and the approximate frequency as input into the mathematical fit to identify the one or more candidate peaks. In this example, both the first threshold and the second threshold may be determined based on values within the frequency spectrum associated with the spectral data.

At step 1430, the process 1400 includes identifying, based on the executed mathematical fit, one or more candidate peaks in the spectral data for a time interval. The time interval may be about 2 minutes to about 6 days; about 10 minutes to about 30 minutes; about 30 minutes to about 1 hour; about 1 hour to about 2 hours; about 2 hours to about 4 hours; about 4 hours to about 8 hours; about 8 hours to about 12 hours; about 12 hours to about 24 hours; about 24 hours to about 30 hours; about 30 hours to about 36 hours; about 36 hours to about 40 hours; about 40 hours to about 48 hours; about 48 hours to about 60 hours; about 60 hours to about 72 hours; about 72 hours to about 80 hours; about 80 hours to about 90 hours; about 90 hours to about 96 hours; about 96 hours to about 108 hours; about 108 hours to about 120 hours; about 10 hours to about 132 hours; or about 132 hours to about 144 hours.

Identifying the one or more candidate peaks in the spectral data may include identifying points within the spectral data that are above the second threshold by executing one or more of: a peak detector that imposes constraints including consecutive values above the first threshold, and a peak detector that provides smoothing prior to identifying points within the spectral data that are above the second threshold.

In some embodiments, the process 1400 may be iteratively performed where each iteration uses a different fitting techniques optimized for identifying candidate peaks having differing widths. Example fitting techniques may include, but are not limited to one or more of: a spectrum smoothing filter, a peak width range thresholding filter, and a Gaussian fitting.

In some embodiments, the identification of the one or more candidate peaks may include identifying each candidate peak and removing the respective signal associated with the candidate peak from the spectral data. For example, the processor 422 may identify a first of the one or more candidate peaks that represents a largest amplitude of each of the one or more peaks in the spectral data. Next, the processor 422 may remove the first of the one or more candidate peaks from the spectral data. The processor 422 may then iteratively perform the method of claim 1 described herein (i.e., process 1400) to identify a second of the one or more candidate peaks, a third candidate peak, a fourth candidate peak, and so on and may iteratively remove each identified peak before moving to the next candidate peak.

At step 1440, the process 1400 includes determining, for the one or more candidate peaks, a plurality of parameters that quantify underlying rhythmic activity of one or more GI organ of the GI tract. The plurality of parameters may include one or more of a center frequency of the one or more candidate peaks, a baseline value of the one or more candidate peaks, a peak width of the one or more candidate peaks, an amplitude of the one or more candidate peaks, and a height of the peak above the baseline value associated with the respective one or more candidate peaks.

At step 1450, the process 1400 includes selecting, based on the determined plurality of parameters and the mathematical fit, at least one of the one or more candidate peaks to represent activity of the GI tract during the time interval. The selection may be based on a number of predefined rules or processes. The rules may include a logical AND of passing or failing the cuts previously mentioned, and/or may include the absolute peak height, ratio of peak height to background level, separation in frequency from another candidate peak, separation in frequency from the edge of the frequency range, residual errors of the mathematical fit, agreement of the frequency and amplitude of the mathematical fit with those parameters used as target input to the fit, and/or the peak width.

In some embodiments, the process 1400 may further include identifying the one or more candidate peaks, removing each of the one or more peaks from the spectral data resulting in background signal, generating an average value range of the background signal, determining a difference between the average value range and a predefined average background level, and generating, based on the determined difference, a normalization factor corresponding to one or more physiological features. Next, the process 1400 may include applying the normalization factor to the spectral data associated with subjects exhibiting one or more of the physiological features including at least one of: subject girth, subject skin condition, subject health condition, subject muscle condition, and subject GI tract anomalies. The generated normalization factor may be used to correct the prior mathematical fit according to the one or more physiological features.

In some embodiments, the process 1400 may assess candidate peaks across more than one channel to ensure validity of the peak. For example, multiple channels of data may be acquired simultaneously using two or more sets of electrodes (e.g., on a single patch or on multiple patches) operating simultaneously. A further cut (e.g., removal of a peak) may be applied based on ensuring that peaks of effectively the same frequency appear in the same time interval on two, or more, such channels, in order to increase the confidence that the peaks so detected are associated with rhythmic signals from the GI tract and not the accidental result of artifacts or random noise. In some embodiments, additional rules may be applied in the case of multiple patches. Such rules may indicate that multiple channel types of cuts ensure the allowable channels in a combination belong to signals originated from the same patch to specify that the geometric orientation of the electrode pairs providing the time series data is the same, i.e. either vertical or horizontal as additional discrimination against accidental peaks. For example, when the spectral data includes multiple channels and represents time series data obtained from at least two sets of electrodes placed on an abdominal region of a subject, data may be captured simultaneously from the at least two sets of electrodes. For each set of electrodes the process 1400 may further include executing the mathematical fit of the spectral data based on the at least one shaping function, identifying, based on the executed mathematical fit, a set of candidate peaks in the spectral data for a time interval, determining, for the set of candidate peaks, a plurality of parameters that quantify underlying rhythmic activity, and selecting, based on the determined plurality of parameters and the mathematical fit, at least one of the set of candidate peaks. Further, the process 1400 may include comparing the selected set of candidate peaks of the first set of electrodes to the selected set of candidate peaks of the second set of electrodes. In response to determining, based on the comparison, that the selected set of candidate peaks of the first set of electrodes and the selected set of candidate peaks of the second set of electrodes appear in the same time interval on two or more of the multiple channels, the process 1400 may include increasing the confidence that the selected sets of candidate peaks represent valid activity of the GI tract.

### Multiple Cleanup Levels

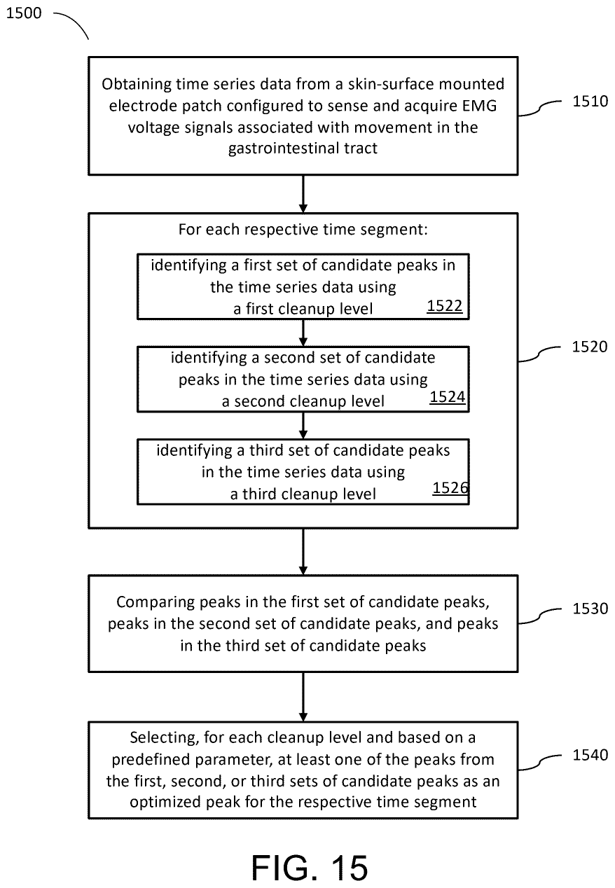

FIG. 15 is a flowchart of an example process 1500 for analyzing spectral peaks associated with movement in a GI tract. The process 1500 may be performed by one or more of the device 400, system 10, and/or another computing device 160A, 160B and/or 160C. In some embodiments, the process 1500 is performed on computing device 160A, 160B, or 160C using data captured by sensor circuit 402, and/or other circuit of device 400. In some embodiments, the process 1500 is performed on processor 422 of device 400.

Because the presence of true rhythmic signals that are particularly strong can mask the presence of lower strength in the spectrum, further cleanup to lower cleanup levels can reveal these lower level signal peaks. It is desirable to include all peaks in the final analysis but no single cleanup level allows this. A solution is to process the data set multiple times, each at a different cleanup level. The system 10 may perform a complete analysis multiple times using different input files for any number of cleanup levels. The input files all begin with the same data set, but are pre-processed with different cleanup levels. For example, each cleanup level sets a unique maximum amplitude of any data point in the time series.

In one non-limiting example, the cleanup levels may be 100 k ADC counts, 50 k ADC counts, 35 k ADC counts, 20 k ADC counts, 10 k ADC counts, and 5 k ADC counts. The peak results may be saved for each run and at the end of this phase of processing compared in each time segment and in each data channel, across the result files. All peaks found across the result files may be combined in an optimized result file. Typically, a given peak frequency will appear in multiple cleanup result files at essentially the same frequency but at different peak heights or areas, in which case only a best peak will be selected for the optimized file. The best peak may be the one with the largest peak height, largest area, or some other parameter such as narrowest width or lowest Gaussian residual.

Processing the data (i.e., input files) across multiple cleanup levels where it is expected that the same peak will appear in multiple such files allows for an additional cut to be made to further improve the likelihood that a peak is valid and not a result of noise, by requiring that it appears in two or more files in order to be included in the optimized result file.

At step 1510, the process 1500 includes obtaining time series data from a skin-surface mounted electrode patch configured to sense and acquire EMG voltage signals associated with movement in the GI tract. The time series data may be obtained for a plurality of time segments over a plurality of channels.

At step 1520, the process 1500 includes for each respective time segment, identifying candidate peaks. For example, at step 1522, the process 1500 includes identifying a first set of candidate peaks in the time series data using a first cleanup level. At step 1524, the process 1500 includes identifying a second set of candidate peaks in the time series data using a second cleanup level. At step 1526, the process includes identifying a third set of candidate peaks in the time series data using a third cleanup level. In this example, the first cleanup level may be about 100,000 analog-to-digital converter (ADC) counts. The second cleanup level may be about 50,000 ADC counts. The third cleanup level may be about 35,000 ADC counts. The results from each cleanup level may provide value beyond their contribution to the optimized result. When both strong and weak peaks exist simultaneously, or when the noise level is such that the smallest peak are not detectable at cleanup levels but do emerge at the lower cleanup levels, their contribution to the total motor activity in the optimized file is minimal. Yet the fact that the signals exist at the lower cleanup levels carries physiological significance. For example in gastroparesis patients, one possible cause in a given patient is a lack of ICC, which provide the electrical pacemaker signal that stimulates muscular contractions. If it is observed that there are indeed signals, however small, at the about 3 cpm frequency these cells produce, then the existence of the cells is confirmed. Practically speaking this means that a gastric motility stimulant has something to work with and therefore might be effective, in contrast to the case where the ICC are substantially diminished.

At step 1530, the process 1500 includes comparing, for each time segment in the plurality of time segments, peaks in the first set of candidate peaks to peaks in the second set of candidate peaks and peaks in the third set of candidate peaks.

At step 1540, the process 1500 includes selecting, for each cleanup level and based on the comparison of the first, second, and third sets of candidate peaks and a predefined parameter, at least one of the peaks from the first, second, or third sets of candidate peaks as an optimized peak for the respective time segment. Example predefined parameters may include at least one of: a maximum peak amplitude, a maximum peak area, a minimum peak width, or a minimum Gaussian residual.

In some embodiments, the process 1500 further includes generating a normalization factor for at least one of the first, second, or third set of candidate peaks and normalizing an amplitude feature in the time series data by applying the generated normalization factor to the time series data. The normalization factor may be derived from a single cleanup file to avoid biases in the data. Providing a normalization scheme that adjusts the overall amplitude of the spectra based on the amplitude in a reference section of the frequency range that is nominally free of peaks, may compensate for the attenuation in signal strength arising from difference in body mass, skin condition and other influencing factors. The normalization scheme may be based on a set of correction values, constituting a two dimensional array of values with dimensions of time on one axis and channel number on the other axis, such values derived from a single cleanup file, so as to avoid biases introduced by the process of cleanup in the reference frequency range used for normalization.

In some embodiments, generating the normalization factor includes identifying a quiet section within the time series data where the quiet section includes one or more voltage ranges substantially free from any apparent valid GI tract signals, any apparent artifacts, or aberrant data patterns, calculating a summed energy of voltage signals contained in the quiet section, comparing the summed energy in the quiet section to a summed energy of a reference spectrum over the same one or more voltage ranges, and determining the data normalization factor based on the comparison.

In some embodiments, the process 1500 further includes, for each respective time segment: identifying a fourth set of candidate peaks in the time series data using a fourth cleanup level, identifying a fifth set of candidate peaks in the time series data using a fifth cleanup level, and identifying a sixth set of candidate peaks in the time series data using a sixth cleanup level. The process 1500 may then compare, for each time segment in the plurality of time segments, peaks in the first set of candidate peaks to peaks in the second set of candidate peaks, peaks in the third set of candidate peaks, peaks in the fourth set of candidate peaks, peaks in the fifth set of candidate peaks, and peaks in the sixth set of candidate peaks in order to select, for each cleanup level and based on the comparison and a predefined parameter, at least one of the peaks from the first, second, third, fourth, fifth, or sixth, sets of candidate peaks as an optimized peak for the respective time segment. In this example, the fourth cleanup level is about 20,000 ADC counts, the fifth cleanup level is about 10,000 ADC counts, and the sixth cleanup level is about 5,000 ADC counts.

In some embodiments, parameters other than cleanup levels may be used as selection criterion for determining a valid peak in the spectral data. For example, a degree of smoothing filter to apply to the spectral data may be used as a parameter when carrying out signal cleanup. In another example, optimizing the detection of narrow peaks may be used as a parameter when carrying out signal cleanup. In yet another example, optimizing the detection of broader peaks may be used as a parameter when carrying out signal cleanup. Example parameters that may be used to generate additional result files that may be input into the optimization process may include an amount of filtering of the spectrum and the widths of the acceptable peaks, for example, to allow for very broad peaks or very narrow ones that are excluded by the standard processing and cuts. This is analogous to the optimization across different cleanup levels that occurs after peak processing on each file, but the filtering may occur during the peak processing on each file. For example, the system 10 may process data in the about 1 cpm to about 5 cpm range (or other range) a second or a third time with different parameters (e.g., filtering, width targets, etc.) to capture peaks that were missed the first time because the peaks were too broad or too narrow. In some embodiments, the system 10 may additionally use different range limits and examine all detected peaks in a time segment, on a given channel, and eliminate multiple copies of what is the same peak (based on frequency) in favor of a best peak.

### Report and Data Presentation

The system 10 may be configured to generate reports and/or output to present data determined using data obtained from one or more devices 400, for example. For example, the system 10 may generate and present test results to physicians, clinicians, and/or patients in a report. The report generally includes answers to questions about the patients' GI motility performance and specific information that is efficient to translate into therapeutic decisions and/or further diagnostic actions. Common vehicles for presentation may include, for example, a representation of the spectra over time, dot plots of detected frequencies versus time, amplitudes in specific frequency bands or as assigned to different organs versus time, and similar representations of basic results. Derived parameters can be useful and may include a ratio of postprandial to pre-prandial activity in a given organ, where the timing of meals is known. The peak histogram, (e.g., a GutPrint® plot) may be generated as a report to depict a representation of the frequencies of all the detected peaks in a particular test. The peak histogram may depict a spectrum-like plot of intensity versus frequency, but unlike a conventional spectrum which has a noise baseline, each entry in the peak histogram is from a detected and discriminated peak, resulting in an improved presentation of spectral data that is related to rhythmic activity.

The nature of the GI tract and its diurnal cycle is such that the shortest time period in which the peak histogram described herein is reproducible is about 24 hours. That is, successive 24 hour peak histograms tend to be similar to one another, but any shorter duration, be it for 1, 2, 4, 8, or 12 hours, does not resemble the one preceding or following it. During resting, particularly during sleep, the gut behaves differently than during waking. For example, there is a higher level of high frequency (˜14 to 18 cpm) colonic activity during the night. Peak histograms constructed from just waking or resting/sleeping hours carry different patterns that are instructive as to the patient's gut performance. In particular, a low ratio of night to day colon activity has been associated with constipation. Reproducibility of the daily peak histogram plot also carries physiological significance; the day to day variation of activity at certain frequencies associated with the stomach and intestines, whether intentionally induced by diet or drug intervention or not, informs the clinician about the health of the patient's gut motility. The variance of the daily peak histogram plots as calculated by a histogram bin to bin standard deviation and mean and median values of those calculations is a useful measure.

In general, the weighted and unweighted peak histograms exhibit different levels of reproducibility with the unweighted having improved reproducibility. This may be linked to the mechanisms of control of the rhythmic activity through the central and enteric nervous systems; the number of cycles per day at each frequency is more consistent than the strength of the contractions. Hence, a metric that evaluates the reproducibility in both cases and the ratio from weighted to unweighted is a useful parameter. In general, a typical peak histogram has meta peaks at the typical common peak frequencies which can be associated with organ specific activity, for example the stomach at 3 cpm. However, signals in the regions between the meta peaks contain information on true rhythmic activity rather than the background noise one sees in simple spectra, and thus may carry clinical significance. Report parameters can be constructed to convey this information economically and intuitively. For example, the ratio of activity in the narrow range associated with the stomach (e.g., about 2.5 to about 3.5 cpm) as compared to the broader 1 cpm to 5 cpm range.

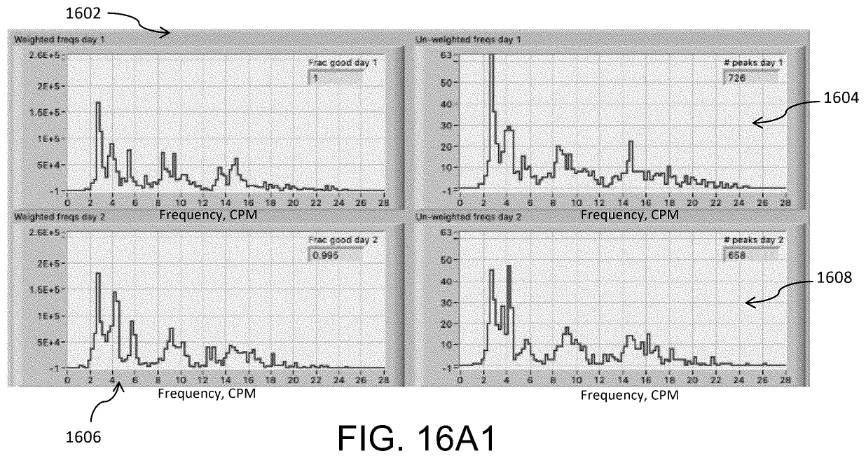

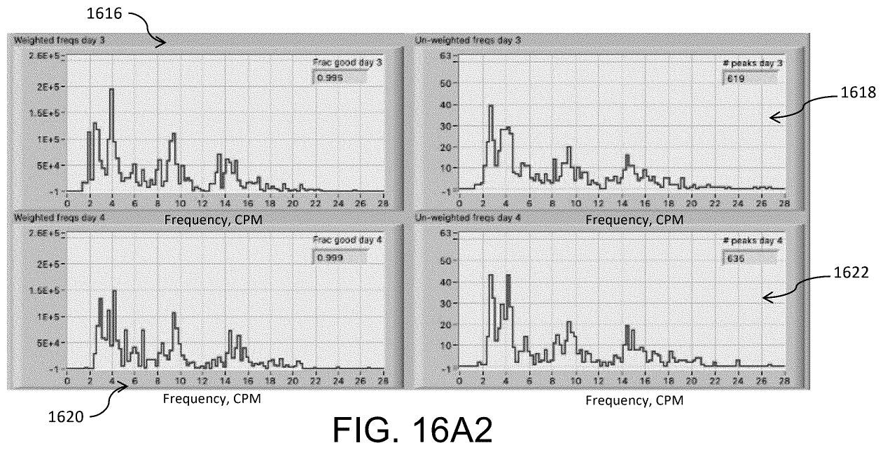

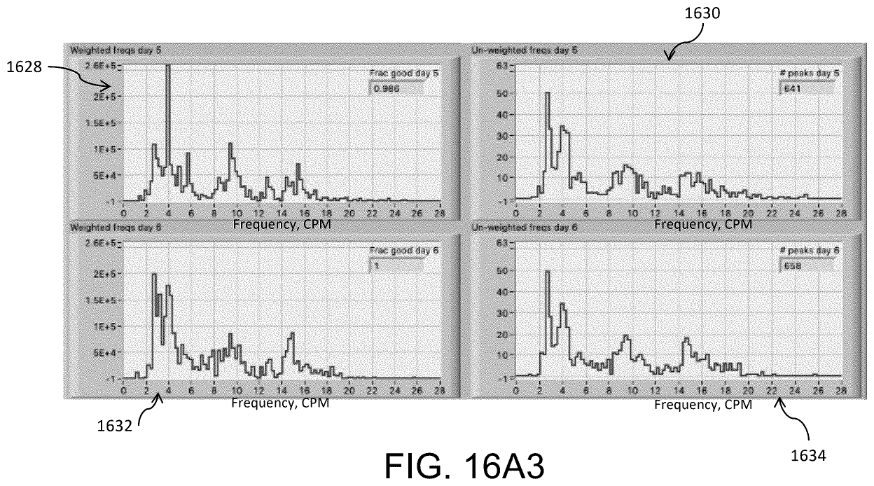

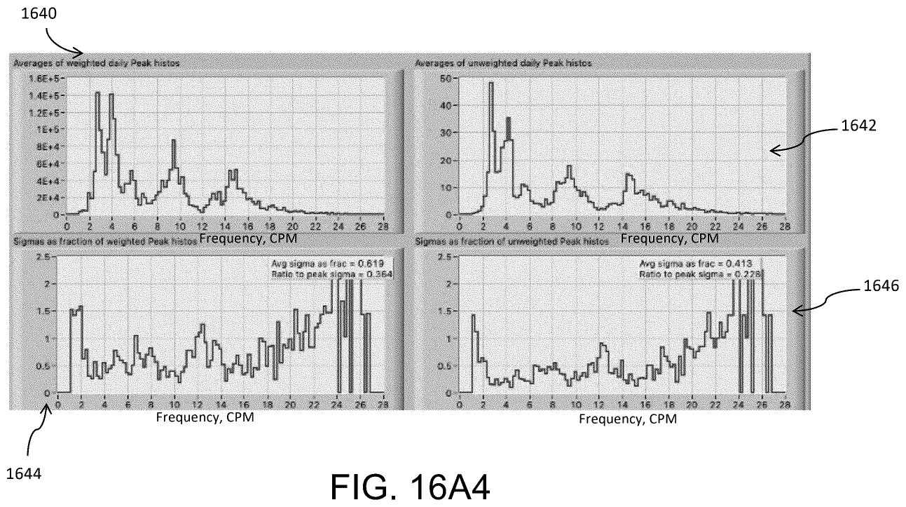

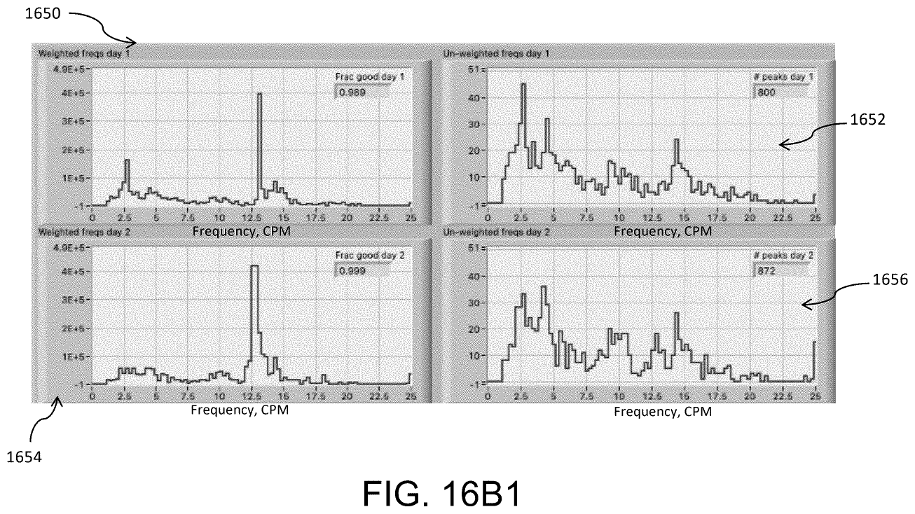

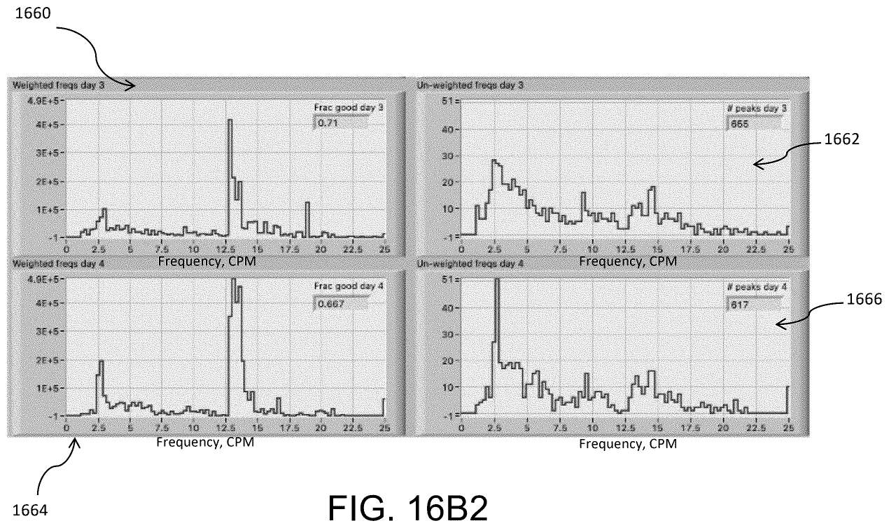

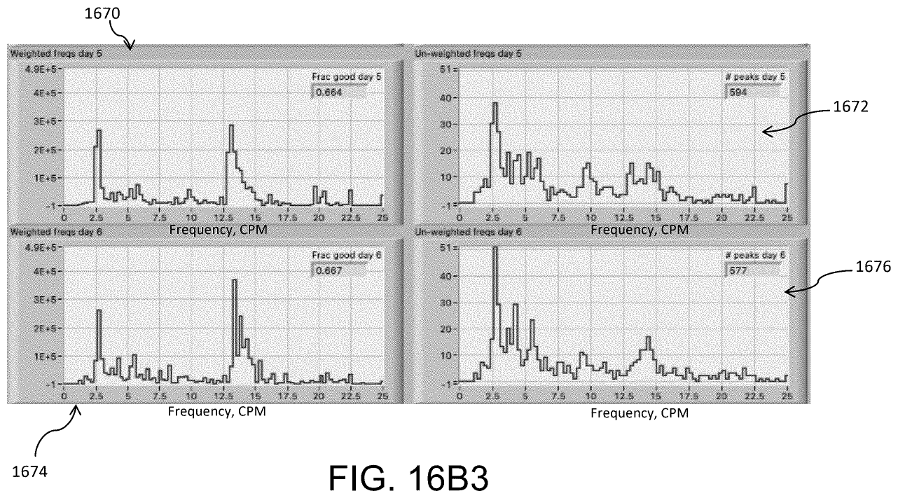

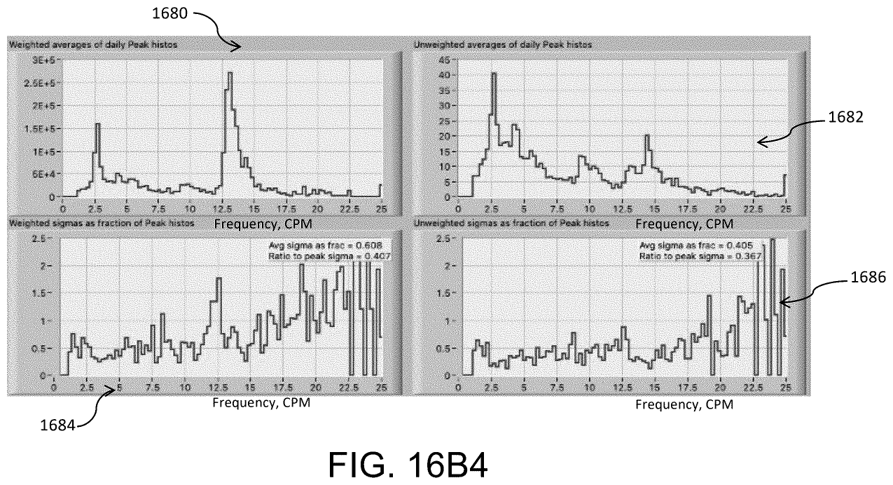

FIGS. 16A1-16A4 illustrate a set of weighted and unweighted daily peak histograms. Each graph depicts values representing the averages in each bin and a histogram representing the standard deviations in each bin, for a test in which the daily variation is relatively small. Daily peak histograms are arranged vertically, oldest (e.g., FIG. 16A1 at the top down to FIG. 16A3, for both weighted (left column) and unweighted (right column) peaks.

For this user's test the day to day behavior is relatively consistent. The unweighted histograms have a lower standard deviation than the weighted, which can be interpreted physiologically as suggesting that the strength of muscular contractions of the GI organs varied more than the number of such contractions, demonstrating the usefulness of such a metric.

FIG. 16A1 includes a first graph 1602 depicting a weighted daily peak histogram representing a first day of measurements for a user and a second graph 1604 depicting a corresponding first day in an unweighted histogram. FIG. 16A1 further includes a third graph 1606 depicting a weighted daily peak histogram representing a second day and a fourth graph 1608 depicting a corresponding second day in an unweighted histogram.

FIG. 16A2 includes a fifth graph 1616 depicting a weighted daily peak histogram representing a third day of measurements for a user and a sixth graph 1604 depicting a corresponding third day in an unweighted histogram. FIG. 16A2 further includes a seventh graph 1620 depicting a weighted daily peak histogram representing a fourth day and an eighth graph 1622 depicting a corresponding fourth day in an unweighted histogram.

FIG. 16A3 includes a ninth graph 1628 depicting a weighted daily peak histogram representing a fifth day of measurements for a user and a tenth graph 1630 depicting a corresponding fifth day in an unweighted histogram. FIG. 16A3 further includes an eleventh graph 1632 depicting a weighted daily peak histogram representing a sixth day and a twelfth graph 1634 depicting a corresponding sixth day in an unweighted histogram.

FIG. 16A4 includes an averaged value across peak histograms. In particular, FIG. 16A4 depicts an average spectrum and a bin-by-bin standard deviation. For example, a first graph 1640 representing an average of weighted daily peak histograms is shown beside a second graph 1642 representing an average of unweighted daily peak histograms for the user assessed in day 1 to day 6, as described above. Graphs 1644 and 1646 depict weighted and unweighted, respectively graphed sigma as a fraction of the peak histograms.

FIGS. 16B1-16B4 represent an example of a test where the user had greater day to day variability, particularly near the 3 cpm peak from the stomach. The metric which quantifies this is of interest, for example, in patients who report gastroparesis or functional dyspepsia type symptoms. Conventional diagnostic tests that measure for one day or just a few hours will typically see whatever behavior is happening at that moment, and could lead to treatments that are inappropriate.

FIG. 16B1 includes a first graph 1650 depicting a weighted daily peak histogram representing a first day of measurements for a user and a second graph 1652 depicting a corresponding first day in an unweighted histogram. FIG. 16B1 further includes a third graph 1654 depicting a weighted daily peak histogram representing a second day and a fourth graph 1656 depicting a corresponding second day in an unweighted histogram.

FIG. 16B2 includes a fifth graph 1660 depicting a weighted daily peak histogram representing a third day of measurements for a user and a sixth graph 1662 depicting a corresponding third day in an unweighted histogram. FIG. 16B2 further includes a seventh graph 1664 depicting a weighted daily peak histogram representing a fourth day and an eighth graph 1666 depicting a corresponding fourth day in an unweighted histogram.

FIG. 16B3 includes a ninth graph 1670 depicting a weighted daily peak histogram representing a fifth day of measurements for a user and a tenth graph 1672 depicting a corresponding fifth day in an unweighted histogram. FIG. 16B3 further includes an eleventh graph 1674 depicting a weighted daily peak histogram representing a sixth day and a twelfth graph 1676 depicting a corresponding sixth day in an unweighted histogram.

FIG. 16B4 includes an averaged value across peak histograms. In particular, FIG. 16B4 depicts an average spectrum and a bin-by-bin standard deviation. For example, a first graph 1680 representing an average of weighted daily peak histograms is shown beside a second graph 1682 representing an average of unweighted daily peak histograms for the user assessed in day 1 to day 6, as described above. Graphs 1684 and 1686 depict weighted and unweighted, respectively graphed sigma as a fraction of the peak histograms.

As shown in FIGS. 16B1-16B4, the user exhibited significant day to day variation in the 3 cpm region, which is from the stomach, and which is a sign of a specific pathology in patients with gastroparesis or functional dyspepsia.

In some embodiments, the system 10 may be configured to perform pre- and post-intervention peak histograms, for example, for the first three days and for the last three days of a test, to assess the effect of a medical, dietary, physiological or other intervention intended to have an effect on motility or GI health in general. In some embodiments, the system 10 may be configured to analyze a ratio of narrow stomach peaks from about 1 cpm to about 5 cpm. For example, the system 10 may calculate and present a particular metric as a measure of stomach dysmotility. The metric may be determined by a ratio of activity in a narrow range encompassing the approximately 3 cpm peak (e.g. about 2.5 cpm to about 3.5 cpm), depending on the observed central peak frequency), which may represent normal stomach activity, to the activity in a broader 1 cpm to 5 cpm range which includes dysrhythmic stomach activity or colon activity.

In some embodiments, the system 10 may be configured to determine a ratio of nighttime to daytime colon activity. For example, the system 10 may calculate and present a metric formed by the ratio of night time to daytime motor activity, as a measure of colon motility and a signal of a type of constipation. The delineation of daytime and nighttime hours may be determined in any of several ways, as a nominal predetermined range, as a result of user entries in the associated mobile app, or based on evidence within the data, such as might be calculated by a measure of heart rate.

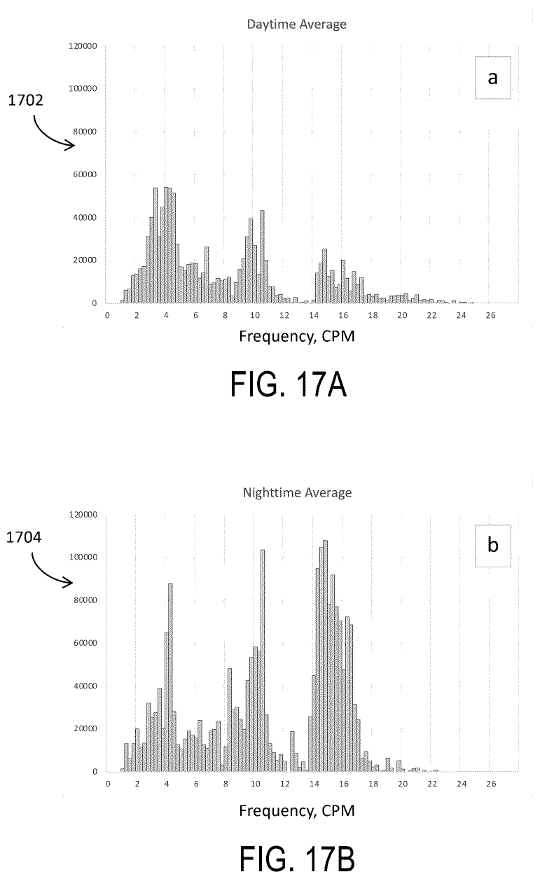

FIG. 17A illustrates a weighted peak histogram 1702 constructed from data acquired during typical daytime (waking) hours for a single subject. FIG. 17B illustrates a weighted peak histogram 1704 constructed from data acquired during typical nighttime (sleeping) hours for the same subject as in FIG. 17A. Differences between sleep and waking are evident across the spectrum, but are noticeably different for sleep and waking in the frequency range between about 14 to about 18 cpm, attributable to activity in the colon (i.e., there is a higher level of activity in about 14 to about 18 cpm range, representing the colon). The area under the curve within a frequency region of interest represents the motor activity, which may be converted to an hourly average or given as a total for the test. A ratio of the colon activity during nighttime (or sleep) to that during the daytime (or waking) provides a metric provided by the patch system described herein that can be used in assessing normal or abnormal behavior of the colon and digestive tract as an aid in understanding the etiology of symptoms such as constipation.

In some embodiments, the system 10 may be configured to calculate and present to a caregiver, for example, histograms of the peaks detected in a peak histogram, both weighted by peak area and unweighted may be included as a graphical metric of the patient's motility characteristics, averaged over a test duration. Further, providing the daily 24 hour versions of these histograms for each day of the test to communicate the variability from day to day, which may provide further insight into the patient's pathophysiology, and as a means of assessing the variance of the full test histograms. Using the daily histograms to calculate that variance and creating a variance or standard deviation graph and average standard deviation value as objective metric of the daily variance.

In some embodiments, the system 10 may be configured to calculate and present to the caregiver frequency versus time dot plots, peak histograms, and/or other graphs for each level of time series data cleanup, to reveal weaker activity at frequencies that may be masked by stronger activity and by random noise, but which may hold physiological significance in the sense of showing that there is indeed electrical activity in the relevant organ, for example, that there are indeed active Interstitial Cells of Cajal present. Knowing that there are ICC present could be important in gastroparesis patients, say to indicate that a motility agent might be effective.

In some embodiments, the system 10 may be configured to compare unweighted to weighted peak histograms with day to day reproducibility being easily viewable. Comparing the unweighted peak histograms to the weighted histograms can provide a decision point for treatment and/or diagnosis. The unweighted peak histograms may be more reproducible day to day suggesting that the number of contractions is the same from day to day but the strength of them differs.

### Heart Rate Analysis and the GI System

In general, the heartbeat is linked to performance of the GI tract directly, through shared signal pathways in the vagus nerve, e.g. fight or flight response, and indirectly. Heart rate variability (HRV), a measure of the short term and moment to moment change in base heart rate, is commonly considered as a potential marker for specific health conditions and overall health. Absolute heart rate, as opposed to HRV, is also useful, for example in determining the patient's current level of activity. The gastrointestinal electrode patch system 10 acquires data at about 5 Hz which is much less than the DAQ rate used for cardiac monitoring (100 Hz and greater), and restricts the possible analysis options to the primary frequency and first harmonic overtone.

One application of the described patch based measurement of heart rate is in using heart rate as a proxy for a resting or a sleeping state, to be applied in calculation of activity in a given organ or in a given frequency range during sleep/resting versus during the awake/active state, as a ratio or difference. Another application is to determine if a patient is under high stress, for example while straining during a bowel movement, which information can be combined with recent prior or concurrent GI motor activity information to identify specific health conditions that could be useful in therapeutic decision making.

A further application of heart rate measurement is in the indication of recovery from surgery as an adjunct measurement in combination with the gastrointestinal data. Immediately after surgery the entire GI tract generally shuts down, with function gradually returning in the small intestine, stomach and colon, typically in that order, over multiple days. Information on how the GI tract is regaining function over the first few days allows physicians to make informed decisions about the patient's feeding schedule and hospital discharge. The system 10 may establish a relationship between heart rate and GI activity and overall recovery through continuous monitoring of heart rate and HRV to provide additional information to inform care decisions.

In some embodiments, data acquired at the lower acquisition rate of a GI electrode system does not support traditional HRV calculations, which typically utilize high resolution tracings of the shape of the heart pulse. However, the long term monitoring capability of multiple days of the systems described herein, versus multiple minutes, supports a calculation of HRV on much longer time scales, based on the nominal ten minute time segments used for spectral analysis of the GI organs (e.g., identified as an HRV-long metric). HRV-long variance can be calculated on time scales using ten minute segments, one hour long segments, or 6, 12, and even 24 hour segments. These metrics have application in post-surgical recovery as well as ambulatory GI disorders. For example correlating a daily HRV metric made up of the variance of hourly averages against a patients report of pain, bloating, nausea or bowel movement frequency, or pre-flare or flare state among IBD or IBS patients, among other possibilities. Heart rate and HRV-long metrics can be applied in assessment of positive or negative effects due to the administration of drugs or diet or lifestyle modifications intended to improve gastrointestinal health, either during a single multi-day test or in separate tests performed before and after the intervention.

In some embodiments, the system 10 may be configured to assess the heart rate as a way to determine sleeping and waking when performing a GI test. For example, the system 10 may be configured to detect and quantify one or more spectral peaks from the heart's fundamental and first harmonic frequencies. The system 10 may track the spectral peaks over time to infer resting versus active periods as a proxy for sleep versus waking, in order to assign the times used for night and day colon activity.

In some embodiments, the system 10 may be configured to assess the heart rate as a way to measure and/or monitor straining. For example, the system 10 may be configured to detect and quantify the one or more spectral peaks from the heart's fundamental and first harmonic frequencies, track the spectral peaks over time to infer a pattern associated with particular stress, such as for example straining during an attempted bowel movement. The system 10 may then corelate these findings with gastrointestinal motor activity in the colon, for example.

In some embodiments, the system 10 may be configured to assess the heart rate to monitor recovery from abdominal surgery. For example, the system 10 may be configured to detect and quantify the one or more spectral peaks from the heart's fundamental and first harmonic frequencies, track the spectral peaks over time during recovery from abdominal or other invasive surgery, to be used in conjunction with gastrointestinal motor activity metrics as a combined metric related to the status of recovery and suitability to initiate feeding and/or discharge from hospital.

In some embodiments, the system 10 may be configured to measure heart rate continuously over multiple days as a new HRV variable. For example, the system 10 may be configured to detect and quantify one or more spectral peaks from the heart's fundamental and first harmonic frequencies, track the spectral peaks over time, and generate a new long period of heart rate variability (HRV-long) parameter based on time scales much longer than traditional HRV, e.g. hourly, multi-hourly, and daily. The long HRV-long metrics may be used to assess and inform recovery from surgery, GI health, and general health.

In some embodiments, the system 10 may be configured to generate and use an HRV-long parameter as metric related to other conditions. For example, the system 10 may be configured to detect and quantify the one or more spectral peaks from the heart's fundamental and first harmonic frequencies, track the spectral peaks over time, generate a new long period of heart rate variability (HRV-long) parameter based on time scales much longer than traditional HRV, apply the HRV-long metrics for assessment of flare status in IBD patients, and/or state of disease in other GI afflictions such as IBS, constipation, diarrhea, and Gastroparesis Syndrome. Additionally, the system 10 may correlate the HRV-long metrics with GI motility measurements in said disease states to provide new diagnostic information that can be used to guide therapy.

In some embodiments, the system 10 may be configured to generate and use HRV-long as a metric in drug and lifestyle modification studies. For example, the system 10 may be configured to detect and quantify one or more spectral peaks from the heart's fundamental and first harmonic frequencies; track the spectral peaks over time, generate a new long period of heart rate variability (HRV-long) parameter based on time scales much longer than traditional HRV, and apply the HRV-long metrics for assessment of positive or negative effects due to the administration of drugs or diet or lifestyle modifications intended to improve GI health, either during a single multi-day test or in separate tests performed before and after the intervention.

Any one or more features of any embodiment disclosed herein can be combined with any one or more other features of any other embodiment, without departing from the scope of the present invention. Further, although the present inventions have been disclosed in the context of certain preferred embodiments and examples, it will be understood by those skilled in the art that the present inventions extend beyond the specifically disclosed embodiments to other alternative embodiments and/or uses and obvious modifications and equivalents thereof. Thus, it is intended that the scope of the present inventions herein disclosed should not be limited by the particular disclosed embodiments described above, but should be determined only by a fair reading of the claims that follow.

The systems and methods and variations thereof can be embodied and/or implemented at least in part as a machine configured to receive a computer-readable, medium storing computer-readable instructions. The instructions are preferably executed by computer-executable components preferably integrated with the system and one or more portions of the processor. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “peak” may include, and is contemplated to include, a plurality of peaks. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for analyzing spectral peaks associated with movement in a gastrointestinal tract, the method comprising:
determining spectral data from electromyographic data originating from smooth muscles associated with one or more organs of the gastrointestinal tract;
executing a mathematical fit of the spectral data based on at least one shaping function;
identifying, based on the executed mathematical fit, one or more candidate peaks in the spectral data for a time interval;
determining, for the one or more candidate peaks, a plurality of parameters that quantify underlying rhythmic activity of one or more gastrointestinal organ of the gastrointestinal tract; and
selecting, based on the determined plurality of parameters and the mathematical fit, at least one of the one or more candidate peaks to represent valid activity of the gastrointestinal tract during the time interval.

2. The method of claim 1, wherein the plurality of parameters comprise:
a center frequency of the one or more candidate peaks,
a baseline value of the one or more candidate peaks,
a peak width of the one or more candidate peaks,
an amplitude of the one or more candidate peaks, and
a height of the peak above the baseline value associated with the respective one or more candidate peaks.

3. The method of claim 1, wherein the time interval ranges from about two minutes to about 4 days.

4. The method of claim 1, wherein the spectral data represents time series data obtained from one or more cutaneous patches placed on an abdominal region of a subject.

5. The method of claim 1, wherein the shaping function is a Gaussian function, a Lorentzian function, or other substantially bell-shaped function.

6. The method of claim 1, wherein the one or more gastrointestinal organ comprises at least one of: a stomach, a small intestine, and a colon.

7. The method of claim 1, wherein executing the mathematical fit comprises:
setting, for the spectral data, a first threshold applicable to identifying an approximate amplitude or width of the one or more candidate peaks;
setting, for the spectral data, a second threshold applicable for identifying an approximate frequency of the one or more candidate peaks; and
using the identified approximate amplitude or approximate width and the approximate frequency as input into the mathematical fit to identify the one or more candidate peaks, wherein both the first threshold and the second threshold are determined based on values within a frequency spectrum associated with the spectral data.

8. The method of claim 7, wherein identifying the one or more candidate peaks in the spectral data further comprises identifying points within the spectral data that are above the second threshold by executing one or more of:
a peak detector that imposes constraints including consecutive values above the first threshold, and
a peak detector that provides smoothing prior to identifying points within the spectral data that are above the second threshold.

9. The method of claim 1, wherein executing the mathematical fit of the spectral data comprises generating, for the spectral data, an optimized background signal associated with the one or more candidate peaks, the optimized background signal resulting in noise reduction of the spectral data in the time interval.

10. The method of claim 1, wherein:
a first of the one or more candidate peaks represents a largest amplitude of each of the one or more peaks;
the first of the one or more candidate peaks is removed from the spectral data; and
performing the method of claim 1 to identify a second of the one or more candidate peaks.

11. The method of claim 1, further comprising:
removing each of the one or more candidate peaks from the spectral data resulting in background signal;
generating an average value range of the background signal;
determining a difference between the average value range and a predefined average background level;
generating, based on the determined difference, a normalization factor corresponding to one or more physiological features;
applying the normalization factor to the spectral data associated with subjects exhibiting one or more of the physiological features, wherein the normalization factor corrects the mathematical fit according to the one or more physiological features.

12. The method of claim 11, wherein the one or more physiological features are selected from at least one of: subject girth, subject skin condition, subject health condition, subject muscle condition, and subject gastrointestinal tract anomalies.

13. The method of claim 1, wherein the method of claim 1 is iteratively performed and each iteration uses a different fitting techniques optimized for identifying candidate peaks having differing widths.

14. The method of claim 13, wherein the fitting techniques comprise one or more of: a spectrum smoothing filter, a peak width range thresholding filter, and a Gaussian fitting.

15. The method of claim 1, wherein:
the spectral data comprises multiple channels and represents time series data obtained from at least two sets of electrodes placed on an abdominal region of a subject and configured to simultaneously capture data; and
for each set of electrodes the method further comprises:
executing the mathematical fit of the spectral data based on the at least one shaping function;
identifying, based on the executed mathematical fit, a set of candidate peaks in the spectral data for a time interval;
determining, for the set of candidate peaks, a plurality of parameters that quantify underlying rhythmic activity; and
selecting, based on the determined plurality of parameters and the mathematical fit, at least one of the set of candidate peaks;
comparing the selected set of candidate peaks of a first set of electrodes, in the at least two sets of electrodes, to the selected set of candidate peaks of a second set of electrodes in the at least two sets of electrodes;
in response to determining, based on the comparison, that the selected set of candidate peaks of the first set of electrodes and the selected set of candidate peaks of the second set of electrodes appear within the time interval on two or more of the multiple channels, increasing a confidence level that the selected sets of candidate peaks represent valid activity of the gastrointestinal tract.

16. A method for analyzing spectral peaks associated with movement in a gastrointestinal tract, the method comprising:
obtaining time series data from a skin-surface mounted electrode patch configured to sense and acquire EMG voltage signals associated with movement in the gastrointestinal tract, the time series data being obtained for a plurality of time segments over a plurality of channels;
for each respective time segment:
identifying a first set of candidate peaks in the time series data using a first cleanup level;
identifying a second set of candidate peaks in the time series data using a second cleanup level;
identifying a third set of candidate peaks in the time series data using a third cleanup level;
comparing, for each time segment in the plurality of time segments, peaks in the first set of candidate peaks to peaks in the second set of candidate peaks, and peaks in the third set of candidate peaks; and
selecting, for each cleanup level and based on the comparison and a predefined parameter, at least one of the peaks from the first, second, or third sets of candidate peaks as an optimized peak for the respective time segment.

17. The method of claim 16, wherein the predefined parameter comprises: a maximum peak amplitude, a maximum peak area, a minimum peak width, or a minimum Gaussian residual.

18. The method of claim 16, wherein:
the first cleanup level is about 100,000 analog-to-digital converter (ADC) counts;
the second cleanup level is about 50,000 ADC counts; and
the third cleanup level is about 35,000 ADC counts.

19. The method of claim 16, further comprising for each respective time segment:
identifying a fourth set of candidate peaks in the time series data using a fourth cleanup level;
identifying a fifth set of candidate peaks in the time series data using a fifth cleanup level;
identifying a sixth set of candidate peaks in the time series data using a sixth cleanup level;
comparing, for each time segment in the plurality of time segments, peaks in the first set of candidate peaks to peaks in the second set of candidate peaks, peaks in the third set of candidate peaks, peaks in the fourth set of candidate peaks, peaks in the fifth set of candidate peaks, and peaks in the sixth set of candidate peaks; and
selecting, for each cleanup level and based on the comparison and the predefined parameter, at least one of the peaks from the first, second, third, fourth, fifth, or sixth, sets of candidate peaks as an optimized peak for the respective time segment.

20. The method of claim 19, wherein:
the first cleanup level is about 100,000 analog-to-digital converter (ADC) counts;
the second cleanup level is about 50,000 ADC counts;
the third cleanup level is about 35,000 ADC counts;
the fourth cleanup level is about 20,000 ADC counts;
the fifth cleanup level is about 10,000 ADC counts; and
the sixth cleanup level is about 5,000 ADC counts.

21. The method of claim 16, further comprising:
generating a normalization factor for at least one of the first, second, or third set of candidate peaks;
normalizing an amplitude feature in the time series data by applying the generated normalization factor to the time series data.

22. A system for analyzing spectral peaks associated with movement in a gastrointestinal tract, the system comprising:
at least one electrode patch mounted on a skin surface of a patient;
at least one processor communicatively coupled to the at least one patch, the at least one processor being configured to characterize parameters of peaks in a frequency spectrum of a gastrointestinal EMG data set acquired from the at least one electrode patch, the characterization comprising:
determining spectral data from electromyographic data captured by the at least one electrode patch and originating from smooth muscles associated with one or more organs of the gastrointestinal tract;
executing a mathematical fit of the spectral data based on at least one shaping function;
identifying, based on the executed mathematical fit, one or more candidate peaks in the spectral data for a time interval;
determining, for the one or more candidate peaks, a plurality of parameters that quantify underlying rhythmic activity of one or more gastrointestinal organ of the gastrointestinal tract; and
selecting, based on the determined plurality of parameters and the mathematical fit, at least one of the one or more candidate peaks to represent valid activity of the gastrointestinal tract during the time interval.

23. The system of claim 22, wherein the plurality of parameters comprise:
a center frequency of the one or more candidate peaks,
a baseline value of the one or more candidate peaks,
a peak width of the one or more candidate peaks,
an amplitude of the one or more candidate peaks, and
a height of the peak above the baseline value associated with the respective one or more candidate peaks.

24. The system of claim 22, wherein the time interval ranges from about two minutes to about 4 days.

25. The system of claim 22, wherein the spectral data represents time series data obtained from one or more cutaneous patches placed on an abdominal region of a subject.

26. The system of claim 22, wherein the shaping function is a Gaussian function, a Lorentzian function, or other substantially bell-shaped function.

27. The system of claim 22, wherein the one or more gastrointestinal organ comprises at least one of: a stomach, a small intestine, and a colon.

28. The system of claim 22, wherein executing the mathematical fit comprises:
setting, for the spectral data, a first threshold applicable to identifying an approximate amplitude or width of the one or more candidate peaks;
setting, for the spectral data, a second threshold applicable for identifying an approximate frequency of the one or more candidate peaks; and
using the identified approximate amplitude or approximate width and the approximate frequency as input into the mathematical fit to identify the one or more candidate peaks, wherein both the first threshold and the second threshold are determined based on values within a frequency spectrum associated with the spectral data.

29. The system of claim 28, wherein identifying the one or more candidate peaks in the spectral data further comprises identifying points within the spectral data that are above the second threshold by executing one or more of:
a peak detector that imposes constraints including consecutive values above the first threshold, and
a peak detector that provides smoothing prior to identifying points within the spectral data that are above the second threshold.

30. The system of claim 22, wherein executing the mathematical fit of the spectral data comprises generating, for the spectral data, an optimized background signal associated with the one or more candidate peaks, the optimized background signal resulting in noise reduction of the spectral data in the time interval.

31. The system of claim 22, wherein:
a first of the one or more candidate peaks represents a largest amplitude of each of the one or more peaks;
the first of the one or more candidate peaks is removed from the spectral data; and
performing the steps in the system of claim 1 to identify a second of the one or more candidate peaks.

32. The system of claim 22, further comprising:
removing each of the one or more candidate peaks from the spectral data resulting in background signal;
generating an average value range of the background signal;
determining a difference between the average value range and a predefined average background level;
generating, based on the determined difference, a normalization factor corresponding to one or more physiological features;
applying the normalization factor to the spectral data associated with subjects exhibiting one or more of the physiological features, wherein the normalization factor corrects the mathematical fit according to the one or more physiological features.

33. The system of claim 32, wherein the one or more physiological features are selected from at least one of: subject girth, subject skin condition, subject health condition, subject muscle condition, and subject gastrointestinal tract anomalies.

34. The system of claim 22, further comprising iteratively performing the determining of the spectral data, the execution of the mathematical fit, the identifying of the one or more candidate peaks, the determining of the plurality of parameters, and the selecting of at least one of the one or more candidate peaks, wherein each iteration uses a different fitting technique optimized for identifying candidate peaks having differing widths.

35. The system of claim 34, wherein the fitting techniques comprise one or more of: a spectrum smoothing filter, a peak width range thresholding filter, and a Gaussian fitting.

36. The system of claim 22, wherein:
the spectral data comprises multiple channels and represents time series data obtained from at least two sets of electrodes placed on an abdominal region of a subject and configured to simultaneously capture data; and
for each set of electrodes:
executing the mathematical fit of the spectral data based on the at least one shaping function;
identifying, based on the executed mathematical fit, a set of candidate peaks in the spectral data for a time interval;
determining, for the set of candidate peaks, a plurality of parameters that quantify underlying rhythmic activity; and
selecting, based on the determined plurality of parameters and the mathematical fit, at least one of the set of candidate peaks;
comparing the selected set of candidate peaks of a first set of electrodes, in the at least two sets of electrodes, to the selected set of candidate peaks of a second set of electrodes in the at least two sets of electrodes;
in response to determining, based on the comparison, that the selected set of candidate peaks of the first set of electrodes and the selected set of candidate peaks of the second set of electrodes appear within the time interval on two or more of the multiple channels, increasing a confidence level that the selected sets of candidate peaks represent valid activity of the gastrointestinal tract.

37. A system for analyzing spectral peaks associated with movement in a gastrointestinal tract, the system comprising:
at least one electrode patch mounted on a skin surface of a patient;
at least one processor communicatively coupled to the at least one patch, the at least one processor being configured to characterize parameters of peaks in a frequency spectrum of a gastrointestinal EMG data set acquired from the at least one electrode patch, the characterization comprising:
obtaining time series data from a skin-surface mounted electrode patch configured to sense and acquire EMG voltage signals associated with movement in the gastrointestinal tract, the time series data being obtained for a plurality of time segments over a plurality of channels;
for each respective time segment:
identifying a first set of candidate peaks in the time series data using a first cleanup level;
identifying a second set of candidate peaks in the time series data using a second cleanup level;
identifying a third set of candidate peaks in the time series data using a third cleanup level;
comparing, for each time segment in the plurality of time segments, peaks in the first set of candidate peaks to peaks in the second set of candidate peaks, and peaks in the third set of candidate peaks; and
selecting, for each cleanup level and based on the comparison and a predefined parameter, at least one of the peaks from the first, second, or third sets of candidate peaks as an optimized peak for the respective time segment.

38. The system of claim 37, wherein the predefined parameter comprises: a maximum peak amplitude, a maximum peak area, a minimum peak width, or a minimum Gaussian residual.

39. The system of claim 37, wherein:
the first cleanup level is about 100,000 analog-to-digital converter (ADC) counts;
the second cleanup level is about 50,000 ADC counts; and
the third cleanup level is about 35,000 ADC counts.

40. The system of claim 37, further comprising for each respective time segment:
identifying a fourth set of candidate peaks in the time series data using a fourth cleanup level;
identifying a fifth set of candidate peaks in the time series data using a fifth cleanup level;
identifying a sixth set of candidate peaks in the time series data using a sixth cleanup level;
comparing, for each time segment in the plurality of time segments, peaks in the first set of candidate peaks to peaks in the second set of candidate peaks, peaks in the third set of candidate peaks, peaks in the fourth set of candidate peaks, peaks in the fifth set of candidate peaks, and peaks in the sixth set of candidate peaks; and
selecting, for each cleanup level and based on the comparison and the predefined parameter, at least one of the peaks from the first, second, third, fourth, fifth, or sixth, sets of candidate peaks as an optimized peak for the respective time segment.

41. The system of claim 40, wherein:
the first cleanup level is about 100,000 analog-to-digital converter (ADC) counts;
the second cleanup level is about 50,000 ADC counts;
the third cleanup level is about 35,000 ADC counts;
the fourth cleanup level is about 20,000 ADC counts;
the fifth cleanup level is about 10,000 ADC counts; and
the sixth cleanup level is about 5,000 ADC counts.

42. The system of claim 37, further comprising:
generating a normalization factor for at least one of the first, second, or third set of candidate peaks; and
normalizing an amplitude feature in the time series data by applying the generated normalization factor to the time series data.
